(19) United States
(12) Patent Application Publication
(10) Pub. No.: US 2025/0152862 A1
(43) Pub. Date: May 15, 2025

(54) APPARATUSES AND METHODS FOR CONTROLLING TEMPERATURE IN AN INHALER DEVICE

(71) Applicant: Syqe Medical Ltd., Tel-Aviv (IL)

(72) Inventors: Binyamin SCHWARTZ, Sde Eliezer (IL); Nimrod RESHEF, LeHavim (IL); Aaron SCHORR, Lotem (IL); Tal LUPO, Yavne (IL)

(73) Assignee: Syqe Medical Ltd., Tel-Aviv (IL)

(21) Appl. No.: 19/027,013

(22) Filed: Jan. 17, 2025

Related U.S. Application Data
(63) Continuation of application No. 17/428,706, filed on Aug. 5, 2021.
(60) Provisional application No. 62/802,737, filed on Feb. 8, 2019.

(51) Int. Cl. A61M 11/04; A24F 40/20; A24F 40/46; A24F 40/57; A61M 15/00

(52) U.S. Cl. CPC A61M 11/042; A24F 40/20; A24F 40/46; A24F 40/57; A61M 15/0003; A61M 15/002; A61M 15/0048; A61M 15/0066; A61M 2205/3368; A61M 2205/3653

(57) ABSTRACT

Some embodiments relate to a method for heating for controlled release of at least one substance to be delivered to a user via inhalation, comprising: allowing airflow through a pallet of source material from which the at least one substance is releasable by vaporization; wherein airflow enters the pallet through a first surface and exits the pallet through a second, opposite surface of the pallet; heating a first heating element in contact with the first surface of the pallet according to a first temperature profile; and heating a second heating element in contact with the second surface of the pallet according to a second temperature profile which is different than the first temperature profile.

### RELATED APPLICATIONS

This application is a Continuation of U.S. patent application Ser. No. 17/428,706 filed on Aug. 5, 2021, which is a National Phase of PCT Patent Application No. PCT/IL2020/050151 having International filing date of Feb. 6, 2020, which claims the benefit of priority under 35 USC §119(e) of U.S. Provisional Patent Application No. 62/802,737 filed on Feb. 8, 2019. The contents of the above applications are all incorporated by reference as if fully set forth herein in their entirety.

### FIELD AND BACKGROUND OF THE INVENTION

The present disclosure, in some embodiments thereof, relates to personal inhaler devices and, more particularly, but not exclusively, to controlling temperature in an inhaler device.

### SUMMARY OF THE INVENTION

According to an aspect of some embodiments of the present invention there is provided a method for delivery of a substance to an inhaling user in an inhaler device, comprising during inhalation by the user: heating at least one of a first surface and a second surface of a source material disposed in the inhaler device to a first temperature; reducing the heating of at least one of the first surface and second surfaces of the source material such that its temperature is gradually reduced to a second temperature below the first temperature; wherein the range between the first temperature and the second temperature maintains the source material within 50° C. of a vaporization temperature range of a substance in the source material.

In some embodiments of a delivery method for example as described herein, the range is within 25° C. of the vaporization temperature.

In some embodiments of a delivery method for example as described herein, the range is within 10° C. of the vaporization temperature.

According to a further aspect of some embodiments of the present invention there is provided method for delivery of a substance to an inhaling user in an inhaler device, comprising during inhalation by the user: stabilizing airflow through the source material at least until the airflow is within a predefined set of parameters; commencing heating of the source material unit to a predetermined first temperature, reducing heating at a predetermined rate to attain a second temperature, wherein heating includes controlling, using a controller, the heating of at least one of an upstream surface and a downstream surface of the source material, the surfaces defined as upstream and downstream according to the airflow path through the source material, using at least one heating element according to pre-programmed operational parameters; and terminating heating of the source material unit after attaining the second temperature.

In some embodiments, reducing the heating does not consist of termination of delivery of power to heat the source material.

In some embodiments, the first temperature is below a combustion temperature of the source material.

In some embodiments, the source material comprises a substance to be delivered by the inhaler, and the first temperature is between 5° C. and 50° C. above a vaporization temperature of the substance.

In some embodiments, the second temperature is low enough such that the maximal temperature of the source material does not exceed the first temperature during the heating.

In some embodiments, the source material comprises a substance to be delivered by the inhaler, and the second temperature is between 5° C. and 50° C. below a vaporization temperature of the substance.

In some embodiments, the second temperature is at least 50° C. above room temperature.

In some embodiments, the method further comprises terminating the method without heating commencement if stabilizing does not occur within a predetermined timeframe.

In some embodiments, heating comprises using an electrically resistive heating element.

In some embodiments, the method further comprises stopping heating in the event of a deviation from a selected temperature by at least a predetermined temperature value.

In some embodiments, the predetermined temperature value is at least 2% higher or lower than the selected temperature.

In some embodiments, a temperature is deemed deviate from a selected temperature if the deviation lasts a period of time being at least 1% of the length of the period of temperature reduction.

In some embodiments, a temperature is deemed deviate from a selected temperature if the deviation lasts a period of time being at least 2% of the length of the period of temperature reduction.

In some embodiments, a temperature is deemed deviate from a selected temperature if the deviation lasts a period of time being at least 15 milliseconds long.

In some embodiments, a temperature is deemed deviate from a selected temperature if the deviation lasts a period of time being at least 25 milliseconds long.

In some embodiments, the method further comprises stopping heating in the event of a deviation from a selected airflow parameter by at least a predetermined airflow value.

In some embodiments, the predetermined value is at least 2% higher or lower than the selected airflow parameter.

In some embodiments, an airflow parameter is deemed deviate from a selected airflow parameter if the deviation lasts a period of time being at least 5% of the length of the period of temperature reduction.

In some embodiments, an airflow parameter is deemed deviate from a selected airflow parameter if the deviation lasts a period of time being at least 10% of the length of the period of temperature reduction.

In some embodiments, an airflow parameter is deemed deviate from a selected airflow parameter if the deviation lasts a period of time being at least 50 milliseconds long.

In some embodiments, an airflow parameter is deemed deviate from a selected airflow parameter if the deviation lasts a period of time being at least 70 milliseconds long.

In some embodiments, the method further comprises stopping heating if a selected temperature is not attained.

In some embodiments, the method further comprises after stopping heating of the source material unit allowing airflow through the inhaler, thereby to flush substance residue from the inhaler device.

In some embodiments, the method further comprises after attaining the second temperature, heating the source material unit to reach a third temperature, higher than the first temperature, and then reducing heating to attain a fourth temperature.

In some embodiments, at least one of the first and second temperatures are selected according to a first target temperature related to a vaporization temperature of a first substance and wherein at least one of the third and fourth temperatures are selected according to a second target temperature related to a vaporization temperature of a second substance.

In some embodiments, the first temperature is below a temperature capable of damaging the first substance.

In some embodiments, at least one of the third and fourth temperatures are above a temperature capable of damaging the substance with the lowest vaporization temperature.

In some embodiments, the method further comprises after reaching the second temperature reaching a third temperature lower than the second temperature.

In some embodiments, the method further comprises reducing heating to reach a fourth temperature lower than the third temperature.

In some embodiments, a time period during which the temperature is reduced from the second temperature to the third temperature is shorter than a time period during which the temperature is reduced from the first temperature to the second temperature, and shorter than a time period during which the temperature is reduced from the third temperature to the fourth temperature.

In some embodiments, stabilizing airflow, commencing heating and terminating heating are all performed during an inhalation of the user from the inhaler device.

According to a further aspect of some embodiments of the present invention there is provided an inhaler device for administration of a substance of a source material to a user, comprising:

at least one conductor configured to supply sufficient energy for heating the source material when the source material is present in a use location within the inhaler;

at least one conduit configured for directing airflow through source material when the source material is present in the use location within the inhaler;

at least one sensor configured to obtain at least one of an indication of a temperature of the source material and an indication of a rate of airflow through the source material; and,

a controller operatively connected to the at least one conductor for controlling the heating temperature, the controller configured with pre-programmed operational parameters and according to the indications received from the at least one sensor, the operational parameters configured to perform the method of any of the preceding claims.

In some embodiments, the controller is operatively connected to both the at least one conductor and the at least one conduit for controlling the heating temperature.

In some embodiments, the device further comprises a compensation airflow regulator, including a controllable valve, the valve located downstream from the source material unit.

In some embodiments, the source material is included in a source material unit configured to be operably attached to the inhaler device. In some embodiments, the source material unit is configured to be received within the use location of the inhaler device.

In some embodiments, the inhaler is configured to receive a magazine containing a plurality of interchangeable source material units for providing the inhaler with a series of source material units.

In some embodiments, the at least one conductor is configured to generate and/or transfer energy to at least a part of the source material unit, which is electrically resistive, to thereby heat the source material.

In some embodiments, the source material unit and the inhaler device have separately operable elements for heating the source material.

In some embodiments, the at least one conductor includes an electrode.

In some embodiments, the at least a part of the source material unit which is electrically resistive is formed as a mesh.

According to a further aspect of some embodiments of the present invention there is provided an inhaler device for heating a substance in a source material, comprising: at least one conductor configured to supply sufficient energy for heating the source material when present in a use location to a first temperature; a controller in operative communication with the at least one conductor and programmed to gradually reduce the heating to a second temperature below the first temperature; wherein the programming of the controller includes a range between the first temperature and the second temperature which maintains the source material within 50° C. of a vaporization temperature range of the substance in the source material.

According to a further aspect of some embodiments of the present invention there is provided an inhaler device for controlling the temperature of a source material unit, comprising: a compensation airflow regulator configured with an adjustable valve for stabilizing airflow through the source material when the source material unit is present in a use location within the inhaler device; at least one electrode for conducting a current to at least a portion of the source material unit; a controller in operative communication with the at least one electrode and programmed to control heating of the source material unit to a predetermined first temperature, and then to reduce heating to attain a second temperature.

In some embodiments, the at least a portion of the source material unit which is electrically resistive is disposed upstream or downstream of the source material.

A “conductor” as referred to herein may include an element configured for generating and/or transferring of electrical and/or thermal energy. In some embodiments, the conductor is configured to generate and/or transfer energy at amount sufficient for heating the source material so as to vaporize one or more active substances from the source material. In some embodiments, the conductor conducts electrical current, for example, an electrode. In some embodiments, the conductor conducts heat.

According to a further aspect of some embodiments of the present invention there is provided an inhaler device for administration of a substance of a source material to a user, comprising: means for heating the source material when present in a use location within the inhaler; at least one conduit configured for directing airflow through source material when present in a use location within the inhaler; and, a controller operatively connected to the heating means and the at least one conduit for controlling the heating temperature, the controller configured with pre-programmed operational parameters and feedback from the at least one sensor.

In some embodiments, the means for heating may include a heating element configured in the inhaler. Additionally, or alternatively, the means for heating include a heating element within the source material unit. In some embodiments, the means for heating include a heating assembly, a portion of which is configured within the inhaler, and a portion of which is configured in the source material unit. Optionally, upon loading of the source material unit into the inhaler, the heating assembly portions come in direct (or indirect) contact with each other (e.g. electrical contact) for supplying energy to heat the source material. In an example of a heating assembly, the inhaler comprises a current conducting electrode which contacts an electrically resistive element of the source material unit, e.g., a mesh, which heats up in response to the applying of current, thereby heating the source material. Optionally, a source material unit comprises a plurality of different source materials, each associated with a different heating element (e.g. a mesh), which can be separately addressed.

In some embodiments, the inhaler comprises one or more integrated source material units, for example positioned within the inhaler housing.

According to an aspect of some embodiments there is provided a method for heating for controlled release of at least one substance to be delivered to a user via inhalation, comprising: allowing airflow through a pallet of source material from which the at least one substance is releasable by vaporization; wherein airflow enters the pallet through a first surface and exits the pallet through a second, opposite surface of the pallet; heating a first heating element in contact with the first surface of the pallet according to a first temperature profile; and heating a second heating element in contact with the second surface of the pallet according to a second temperature profile which is different than the first temperature profile.

In some embodiments, the method comprises controlling heating by increasing or reducing a temperature of one or both of the first heating element and the second heating element.

In some embodiments, the first temperature profile comprises heating to a first temperature and maintaining it constant; and the second temperature profile comprises heating to a second temperature and maintaining the temperature constant, the first and second temperatures being different from each other.

In some embodiments, the method comprises controlling heating to maintain at least 85% of the source material within a target temperature range.

In some embodiments, the method comprises modifying heating of one or both of the first and second heating elements in response to a change in the rate of airflow through the pallet.

In some embodiments, the method comprises controlling heating to control at least one of: an amount of substance released and a duration of time over which the substance is released.

In some embodiments, heating of the first and second heating elements is to a temperature that does not fall within a target temperature range of the source material.

In some embodiments, the target temperature range comprises a range within 25° C. of a vaporization temperature of the at least one substance.

In some embodiments, heating of the first and second heating elements is to a temperature that does not cause combustion of the source material.

In some embodiments, allowing airflow comprises allowing airflow in a direction transverse to the first and second surfaces of the pallet.

In some embodiments, the first heating element and the second heating element are portions of a single heating element.

In some embodiments, the single heating element is “U” shaped, and heating comprises conducting electrical current through the “U” shape.

In some embodiments, controlling heating comprises indirectly controlling heating by changing a rate of the airflow through the pallet.

According to an aspect of some embodiments there is provided a heating module useable in an inhaler device configured to receive a source material unit, the source material unit including first and second electrically resistive heating elements in contact with source material, the heating module comprising: at least two electrical contacts shaped and positioned to engage the first and second electrically resistive heating elements of the source material unit when the source material unit is received within the inhaler device; and circuitry for controlling conduction of current by the at least two electrical contacts for heating the first and second heating elements to raise a temperature of at least 85% of the source material to a target temperature; the circuitry configured to control heating of the first heating element to a first temperature and heating of the second heating element to a second temperature different than the first temperature.

In some embodiments, the circuitry is configured to control heating of the first and second heating elements to maintain the heated source material within a range of +/−15% of the target temperature.

In some embodiments, the circuitry is configured to control heating of the first and second heating elements in accordance with a rate of airflow through the source material unit.

In some embodiments, the heating module comprises at least one sensor positioned to measure, when the source material unit is received within the inhaler device, the temperature of at least one of: the first heating element, the second heating element, the source material or portions; the circuitry configured to control heating of the first and second heating elements in response to an indication received from the at least one sensor.

In some embodiments, the circuitry controls heating of the first and second heating elements to raise a temperature of the source material to a temperature range within 10° C. of a vaporization temperature of the at least one substance within less than 2 seconds.

In some embodiments, the circuitry controls heating of the first and second heating elements to stabilize and maintain the source material temperature within in the vaporization temperature range for a time period of 0.5 seconds or longer.

In some embodiments, the first and second heating elements are parts of a single heating element and the circuitry is configured to deliver a similar amount of electric energy to both the first and second heating elements.

According to an aspect of some embodiments there is provided a kit comprising: an inhaler device including a heating module; and a source material unit including first and second electrically resistive heating elements in contact with source material, the source material unit shaped and sized to be received within a housing of the inhaler.

In some embodiments, the source material is in the form of a pallet having a thickness between 0.5-1 mm.

In some embodiments, a surface area of each of first and second opposing surfaces of the pallet which are heated by the first and second heating elements respectively is between 200-300 mm\hat{ }2.

In some embodiments, a weight of the pallet is between 100-150 mg.

In some embodiments, the pallet comprises source material particles dispersed with spaces therebetween through which air is allowed to flow.

According to an aspect of some embodiments there is provided a method for delivering to a user via an inhaler device one or more substances releasable from a source material by vaporization, comprising: heating at least one of a first surface and a second surface of a source material disposed in the inhaler device to a first temperature; reducing heating of the heated at least one of the first surface and second surfaces of the source material such that its temperature is reduced to a second temperature below the first temperature; wherein the range between the first temperature and the second temperature maintains the source material within 50° C. of a vaporization temperature range of a substance in the source material.

In some embodiments, the range is within 25° C. of the vaporization temperature.

In some embodiments, the range is within 10° C. of the vaporization temperature.

In some embodiments, heating and reducing the heating are during an inhalation of a user from the inhaler device.

In some embodiments, the method comprises allowing airflow at a direction perpendicular to the first and the second surfaces.

In some embodiments, a distance between the first and the second surfaces, across the source material, is between 0.2-1.00 millimeter.

In some embodiments, the first temperature is below a combustion temperature of the source material.

In some embodiments, the second temperature is low enough such that the maximal temperature of the source material does not exceed the first temperature during heating.

In some embodiments, the second temperature is at least 50° C. above room temperature.

In some embodiments, heating of the at least one first and second surfaces is by at least one heating element which is an electrically resistive heating element.

In some embodiments, the method further comprises stopping heating in the event of a deviation from a selected temperature by at least a predetermined temperature value.

In some embodiments, the method further comprises, after attaining the second temperature, heating the source material to reach a third temperature, higher than the first temperature, and then reducing heating to attain a fourth temperature.

In some embodiments, at least one of the first and second temperatures are selected according to a first target temperature related to a vaporization temperature of a first substance and wherein at least one of the third and fourth temperatures are selected according to a second target temperature related to a vaporization temperature of a second substance.

In some embodiments, the method further comprises the first temperature is below a temperature capable of damaging the first substance.

According to an aspect of some embodiments there is provided a method of controlling release of at least two substances having different vaporization temperatures from a source material, for delivering the substances to a user by inhalation, comprising: passing airflow through the source material; heating the source material to a first temperature within a range of 25° C. from a vaporization temperature of the first substance to generate release of the first substance; wherein the second substance substantially does not vaporize when heating the source material to the first temperature; and heating the source material to a second temperature within a range of 25° C. from a vaporization temperature of the second substance to generate release of the second substance.

In some embodiments, the method comprises reducing or terminating heating between the first heating and the second heating.

In some embodiments, release of the first substance and the second substance at least partially overlaps in time.

In some embodiments, the second substance is released only a selected time period following release of the first substance.

In some embodiments, passing airflow comprises controlling the airflow rate through the source material.

In some embodiments, heating and passing airflow are controlled to release the first substance and the second substance at a selected ratio.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example, are not necessarily to scale, and are for purposes of illustrative discussion of embodiments of the invention. In this regard, the description taken with the drawings makes apparent to those skilled in the art how embodiments of the invention may be practiced.

In the drawings:

FIG. 1 is a schematic diagram showing the air flow in an inhaler device, according to some embodiments;

FIG. 2 is a block diagram showing components of an inhaler device, according to some embodiments;

FIG. 3 is a perspective, partially-exploded view of a source material unit, according to some embodiments;

FIG. 4 is a cross-sectional view of a source material unit, according to some embodiments;

FIG. 5 is a flowchart of a method for controlling the thermal performance of a source material unit in an inhaler device, according to some embodiments;

FIGS. 6A and 6B are graphs showing multi-step heating methods, according to some embodiments;

FIGS. 7A-B are flowcharts of methods for selecting a temperature profile to control or affect release of one or more substances, according to some embodiments; and

FIGS. 8A-B graphically show examples of substance release in correlation with temperature profiles for example as shown in FIGS. 6A-B, according to some embodiments;

FIG. 9 is a schematic diagram of a heating module for heating a source material, according to some embodiments;

FIG. 10 is a flowchart of a method for controlled heating of source material, in accordance with some embodiments;

FIG. 11 is a graphical representation of a temperature profile of the source material over time, according to some embodiments;

FIGS. 12A-C schematically illustrate an estimated effect of heating a source material pallet from one or two surfaces of the pallet, according to some embodiments;

FIGS. 12D-E graphically compare heating of a source material pallet when there is air flowing through the pallet and when there is no airflow through the pallet, according to some embodiments; and

FIG. 13 is a schematic drawing of an airflow scheme across one or more surfaces of a source material pallet, according to some embodiments.

### DESCRIPTION OF SPECIFIC EMBODIMENTS OF THE INVENTION

The present disclosure, in some embodiments thereof, relates to personal inhaler devices and, more particularly, but not exclusively, to controlling temperature in an inhaler device.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

The term “Source material unit”, as used throughout this specification, optionally refers to a dose cartridge/chip/repository and/or other element which includes or is composed of source material. A source material unit contains a known, measured amount of a source material for the delivery of at least one vaporizable substance associated therewith having a vaporization temperature. For example, the source material may comprise or consist of botanical matter, plant matter, a synthetic carrier and/or an inert carrier (e.g. cellulose or synthetic beads or filaments). The source material may be in or comprise any form or structure compatible with its use, including for example a granulate, powder, beads, filaments, a mesh or perforated material. Optionally, the source material is permeable to air in that it allows a flow of at least 0.5 liter of gas per minute under a pulling vacuum of at least 1-5 kPa.

The term “substance” as referred to herein may include or consist of one or more natural and/or synthetic compounds, molecules, pharmaceuticals, drugs, or the like, that are contained in and/or otherwise associated with or carried by the source material. Optionally, a substance is associated with the source material when in one form and undergoes a change during heating and/or vaporization. For example—a cannabinoid that is present in cannabis in acid form and undergoes decarboxylation when heated (such as from THCA to THC or CBDA to CBD).

A “vaporization temperature” as used herein may mean a temperature or temperature range in which a substance undergoes vaporization. In some embodiments, the vaporization temperature is included as an operational element or parameter of the inhaler (amongst other parameters, such as pressure, time, flow rate, current, and the like).

Generally, the inventors of the present invention surprisingly discovered that the temperatures of a first, upstream surface of the source material unit and a second, downstream surface were significantly different, where upstream and downstream are defined by airflow through the inhaler during use. These temperatures were measured, in accordance with some embodiments, as a temperature of a resistive heating element (e.g. a mesh) contacting each of the surfaces. This detected difference in temperature occurred despite the fact that the source material was in the form of a flattened mass, airflow was along a path being no more than 1 mm thick, and both surfaces were being heated concomitantly by delivery of the same amount of power to both sides. It was discovered that when controlling heating to maintain a temperature of the upstream surface near a target vaporization temperature, the temperature differential may lead to significant overheating of the downstream surface, and that when controlling heating to maintain a temperature of the downstream surface near a target vaporization temperature, the temperature differential may lead to significant under-heating of the upstream surface.

In some embodiments of the invention, and as described in more detail herein, methods and related structures are proposed to heat a first surface of the source material (and/or a filter of the source material adjacent the first surface, and contacting the first surface) to a first temperature being above a target temperature and then have a controlled temperature reduction that ends with a second temperature being below the target temperature.

In some embodiments, the target temperature is the vaporization temperature of a substance intended for delivery by the inhaler. Optionally, the target temperature is a temperature above the vaporization temperature. Optionally, the target temperature is below the vaporization temperature. Optionally, the target temperature is within a selected range which is higher and/or lower than the vaporization temperature. Additionally, or alternatively, the vaporization temperature is a temperature that is below a combustion temperature of the source material or a combustion temperature of a portion of the source material.

The first temperature may be selected to be below a combustion temperature of the source material (or of any portion thereof), but, optionally, above the vaporization temperature of a substance in the source material, and optionally within the range of between 5° C.-50° C. or between 10° C.-30° C. above a target temperature. The second temperature may be low enough such that the maximal temperature of the source material does not exceed the first temperature during the heating. Optionally, the second temperature is between 5° C.-50° C. or between 10° C.-30° C. below a target temperature of the substance.

In some embodiments, the first surface of the source material is the upstream surface. In such embodiments, the first and second temperatures of the first surface are selected such that the temperature of the second (downstream) surface is equal to the temperature of the first surface and/or higher than the temperature of the first surface, but (optionally) lower than a combustion temperature of the source material (or of any portion thereof).

In some embodiments, the first surface of the source material is the downstream surface. In some such embodiments the first and second temperatures of the first surface are selected such that the temperature of the second (upstream) surface or a temperature within the source material is equal to the temperature of the first surface and/or lower than the temperature of the first surface, and is optionally higher than a vaporization temperature of the substance intended for delivery by the inhaler for at least 50%, 70%, 80%, 90%, 95% or the entire duration of this controlled temperature reduction step.

It is noted that a given temperature may be a temperature actually sensed at a given location or a temperature calculated or estimated according to sensing of temperature at the same and/or other locations. Optionally, the given temperature is a temperature sensed during experimentation and/or during actual use of the inhaler device.

As described herein, heating of the upstream surface and/or the source material and/or the downstream surface is intended to exceed and/or attain and/or maintain and/or approximate a target vaporization temperature which is matched to one or more substance located in the source material unit for delivery to a user by the inhaler.

In some embodiments, the source material comprises plant material, for example cannabis and/or tobacco, and an active substance (e.g. THC and/or nicotine) is extracted by heating the plant matter and/or airflow through the plant material. Other examples for plant material include one or more of Cannabis sativa, Cannabis indica, Cannabis ruderalis, Acacia spp., Amanita muscaria, Yage, Atropa belladonna, Areca catechu, Brugmansia spp., Brunfelsia latifolia, Desmanthus illinoensis, Banisteriopsis caapi, Trichocereus spp., Theobroma cacao, Capsicum spp., Cestrum spp., Erythroxylum coca, Solenostemon scutellarioides, Arundo donax, Coffea arabica, Datura spp., Desfontainia spp., Diplopterys cabrerana, Ephedra sinica, Claviceps purpurea, Paullinia cupana, Argyreia nervosa, Hyoscyamus niger, Tabernanthe iboga, Lagochilus inebriens, Justicia pectoralis, Sceletium tortuosum, Piper methysticum, Catha edulis, Mitragyna speciosa, Leonotis leonurus, Nymphaea spp., Nelumbo spp., Sophora secundiflora, Mucuna pruriens, Mandragora officinarum, Mimosa tenuiflora, Ipomoea violacea, Psilocybe spp., Panaeolus spp., Myristica fragrans, Turbina corymbosa, Passiflora incarnata, Lophophora williamsii, Phalaris spp., Duboisia hopwoodii, Papaver somniferum, Psychotria viridis, spp., Salvia divinorum, Combretum quadrangulare, Trichocereus pachanoi, Heimia salicifolia, Stipa robusta, Solandra spp., Hypericum perforatum, Tabernaemontana spp., Camellia sinensis, Nicotiana tabacum, Nicotiana rustica, Virola theidora, Voacanga africana, Lactuca virosa, Artemisia absinthium, Ilex paraguariensis, Anadenanthera spp., Corynanthe yohimbe, Calea zacatechichi, Coffea spp. (Rubiaceae), Sapindaceae spp., Camellia spp., Malvaceae spp., Aquifoliaceae spp., Hoodia spp. Chamomilla recutita, Passiflora incarnate, Camellia sinensis, Mentha piperita, Mentha spicata, Rubus idaeus, Eucalyptus globulus, Lavandula officinalis, Thymus vulgaris, Melissa officinalis, Tobacco, Aloe Vera, Angelica, Anise, Ayahuasca (Banisteriopsis caapi), Barberry, Black Horehound, Blue Lotus, Burdock, Camomille/Chamomile, Caraway, Cat's Claw, Clove, Comfrey, Corn Silk, Couch Grass, Damiana, Damiana, Dandelion, Ephedra, Eucalyptus, Evening Primrose, Fennel, Feverfew, Fringe Tree, Garlic, Ginger, Ginkgo, Ginseng, Goldenrod, Goldenseal, Gotu Kola, Green Tea, Guarana, Hawthorn, Hops, Horsetail, Hyssop, Kola Nut, Kratom, Lavender, Lemon Balm, Licorice, Lion's Tail (Wild Dagga), Maca Root, Marshmallow, Meadowsweet, Milk Thistle, Motherwort, Passion Flower, Passionflower, Peppermint, Prickly Poppy, Purslane, Raspberry Leaf, Red Poppy, Sage, Saw Palmetto, Sida Cordifolia, Sinicuichi (Mayan Sun Opener), Spearmint, Sweet Flag, Syrian Rue (Peganum harmala), Thyme, Turmeric, Valerian, Wild Yam, Wormwood, Yarrow, Yerba Mate, and/or Yohimbe. The dosing botanical substance optionally includes any combination of plant material from this list, and/or other plant material. Optionally, the source material comprises one or more synthetic or extracted drugs added to or applied on carrier material, wherein the added drug and/or the source material may be in the form of or comprise solid material, gel, powder, encapsulated liquid, granulated particles, and/or other forms. In some embodiments, the source material comprises plant material having one or more synthetic or extracted drugs added thereto or applied thereon.

In some embodiments, the upstream surface and the downstream surface comprise a filter or filter-type structure, configured to allow airflow therethrough, but not to allow passage of the source material through (e.g. passage of source material particles). In some embodiments, the airflow passing through the source material contains a produced vapor or aerosol, for example, vapors of substances released from a more upstream portion of the source material.

In some embodiments, the filter includes a plurality of layers and/or portions, at least one configured to maintain the source material and at least one configured to heat the surface. Optionally, the heating of an upstream filter (upstream of the source material being heated) is controlled to indirectly control the heating and/or cooling of a downstream filter (downstream of the source material being heated). In some embodiments, the upstream filter and the downstream filter are of unitary construction, and are included in a single filter structure, for example a structure folded into a “U” shape around the source material in the source material unit to functionally create upstream and downstream filters. In some embodiments, the upstream filter and the downstream filter are different structures, optionally physically separate.

In some embodiments, sensing of the heating of the upstream and downstream surfaces is conducted by at least one temperature sensor for sensing each surface. In some embodiments, sensing of the heating is conducted on the upstream surface or the downstream surface. In some embodiments, sensing is not performed. In some embodiments, heating is performed by at least one heating element that is a component of the inhaler device.

In some embodiments, the heating is performed by a component of the source material unit. Optionally, heating is performed by at least one heating element from both the inhaler device and the source material unit in combination. Optionally, the at least one heating element is or includes at least one electrode and/or a thermally conductive structure like a filter or mesh and/or a structure/component within the source material itself. Optionally, a combination of heating elements includes at least one electrode in the inhaler and at least one electrically conductive element being in thermally conductive contact with the source material or a portion thereof, such that driving an electric current through the electrode to the electrically conductive element causes it to heat, and thereby heat the source material. In some embodiments, the inhaler comprises an electrical contact, for supplying energy sufficient for heating the source material. Optionally, the electrical contact comprises of at least one electrode for conducting a current to an electrically resistive element of the source material unit, to thereby heat the source material. Other optional examples of heating elements which could be used include heating using laser, magnetism (e.g. induction), infrared and microwave, as examples.

In some embodiments, a heating profile of the source material is selected for controlling release of one or more substances from the source material. In some embodiments, more than one vaporizable substance (e.g. 2, 3, 5, 10 substances or intermediate, larger, or smaller amount) are contained in a source material, and their release is at least partially controlled by controlling the temperature to which the source material is heated. By controlling the heating profile, parameters such as the type of substance released, the amount of substance released, a ratio between two or more substances released, a duration of substance release, a relative timing for releasing of two or more substances may be controlled. In some embodiments, the heating profile is selected in accordance with thermal and/or chemical and/or structural properties of the releasable substance(s). For example, a heating profile may be selected to raise the temperature of the source material rapidly, thereby generating release of a first substance at a relatively high rate and/or amount and release of a second substance, optionally having different properties, at a lower rate and/or amount. In another example, a heating profile may be selected to raise the temperature of the source material to a temperature that is within the range of a vaporizing temperature of a first substance; then optionally reduce or terminate heating; then change the temperature to a temperature that is within the range of a vaporizing temperature of a second substance, to generate release of the second substance subsequently and/or partially overlapping and/or a selected time period after releasing of the first substance. In another example, heating is controlled to increase the percentage of the substance being released from the source material, potentially improving the usability.

In some embodiments, an airflow profile through the source material is controlled. Optionally, the airflow profile is synchronized with the heating profile to control and/or affect release of the one or more substances. In an example, the temperature is raised simultaneously to increasing an airflow rate through the source material, to accelerate substance release.

In some embodiments, the heating profile and/or airflow profile are controlled to deliver two or more substances to an inhaling user during a single inhalation of the user.

An aspect of some embodiments relates to controlled heating of a source material through which air is allowed to flow, by setting a heating profile of one or more heating elements of the source material. In some embodiments, two heating elements are placed in thermal communication (optionally, in contact) with two surfaces of a source material pallet. In use, air is allowed to flow, for example, through the first heating element, through the source material of the pallet, and then through the second heating element. In some embodiments, heating of each of the heating elements is controlled by circuitry, which sets parameters of heating (such as a maximal temperature, a heating rate, a heating duration and/or other parameters) according to parameters including, for example, a rate of airflow through the pallet, a thickness of the pallet, a density of the source material, and/or other parameters. In some embodiments, heating of the heating elements is controlled to bring and optionally maintain the source material within a target temperature range. Optionally, the target temperature range is a range in which at least one selected substance vaporizes from the source material.

In some embodiments, heating is controlled to compensate for cooling and/or heating effects caused by the airflow. For example, airflow may cool layers of the source material pallet which are adjacent the airflow entry to the pallet; for example, the flow of air may be heated by the first (upstream) heating element, thereby causing more downstream layers of the pallet to be heated more than upstream layers.

In some embodiments, heating of the heating element(s) is controlled indirectly, for example by changing the airflow, such as by changing the airflow rate and/or direction.

In some embodiments, a modeled temperature distribution in a source material pallet which is heated on opposite sides thereof is used for prediction of the temperature profiles required for heating the source material to the target temperature or range. The modeled temperature distribution, according to some embodiments, takes into account the effects of airflow passing the pallet.

In some embodiments, opposing heating elements are formed as a single unit. In an example, opposing heating elements define the arms of a “U” shaped unit. In some embodiments, electrical current is applied to heat the unit as a single unit. In some embodiments, in the example of a “U” shaped unit, different temperatures develop on each of the opposing heating elements as a result of various conditions including, for example, flow of air (e.g. through the pallet); structural conditions (e.g. device components located in proximity to the heating element); and/or other conditions. Optionally, heating is applied to the single unit such that if the unit was under no effects of the surroundings, both heating elements would have been heated to a similar temperature. Optionally, the bending portion of the “U” shape is heated to a higher temperature, such as due to conduction of heat from both arms.

In some embodiments, closed-loop control of heating is performed. Optionally, indications from one or more temperature sensors and/or from one or more flow rate sensors are received by the control circuitry (e.g. the device controller) and heating of one or both of the heating elements is initiated, increased, reduced, maintained and/or terminated based on the indication(s) received from the sensor(s). In some embodiments, an indication of temperature is received not by a sensor, but, for example, based on impedance/conductivity properties of device circuitry, for example based on the electrical resistance of the heating element.

Alternatively, in some embodiments, heating is not under closed-loop control or based on feedback. In such embodiments, heating may be applied according to one or more predefined profiles. Optionally, the predefined profile defines (optionally for each of the heating elements) a duration of heating, a temperature profile (e.g. a constant temperature or a temperature that varies with time), powering of the heating element. In some embodiments, parameters of a heating profile are determined or calculated according to a database, a look up table, formulas and the like. Optionally, heating profile parameters are determined or calculated based on experimental results.

As referred to herein, heating of a heating element to a certain temperature or according to a temperature profile may include inputting energy sufficient to heat the heating element to that temperature, assuming no flow or air and/or other effects which may increase or reduce the actual temperature of the heating element. In some embodiments, heating a heating element to a certain temperature involves supplying power suitable to raise a temperature of an electrically resistive heating element to the selected temperature. It should be understood that the examples described herein could be applied to any structures which exhibit uneven thermal performance under operating conditions that similarly exist for any source material unit in any inhaler device.

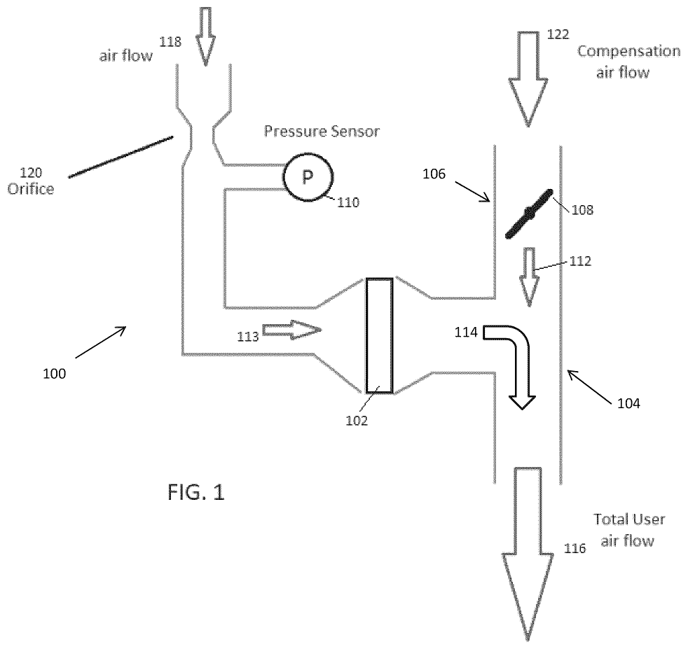

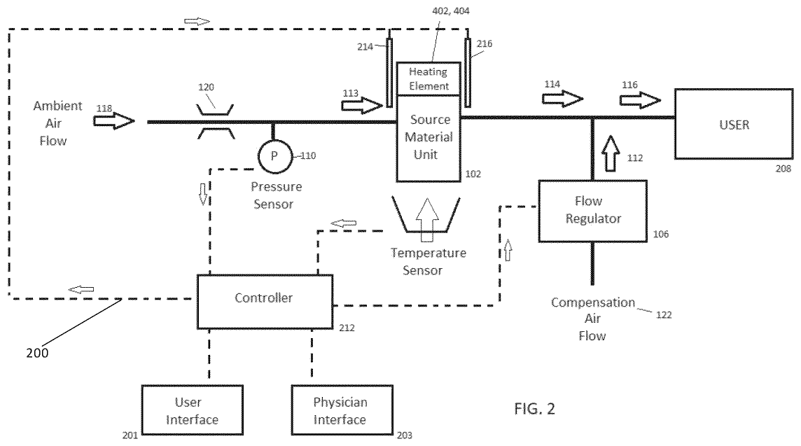

FIG. 1 is a schematic diagram of an inhaler device 100, according to some embodiments, having a source material unit 102 positioned in a use location within the inhaler. An airflow conduit 104, which is operative to deliver substance-imbued airflow to a user 208 (shown and described in more detail with respect to FIG. 2 is included in the inhaler device 100, downstream of source material unit 102. It should be understood that the air flow into the inhaler device 100 stems from the user 208 inhaling on the inhaler device 100 and creating intake air flow 118 into the orifice 120 (which thereafter enters the source material as airflow 113) and, optionally, the compensation airflow regulator 106.

In some embodiments, a compensation airflow regulator 106, for regulating compensation air flow 122, is included additionally to the airflow output through conduit 104 for modifying airflow 116 delivered to the user 208. In some embodiments, the compensation airflow regulator 106 includes a controllable valve 108 which can be open or closed or partially closed to regulate the flow of air 112 into the airflow 114 coming out of the source material unit.

In some embodiments, heating of the source material unit 102 is controlled by a controller 212 (shown and described in more detail with respect to FIG. 2), which controls at least one heating element in accordance with pre-programmed operational parameters. In some embodiments, at least one sensor 110 is used, for example a pressure sensor, to measure and/or sense/detect a parameter indicative of airflow or airflow rate. Optionally, a sensor 110 is positioned near the orifice 120 to detect intake airflow and/or airflow rate.

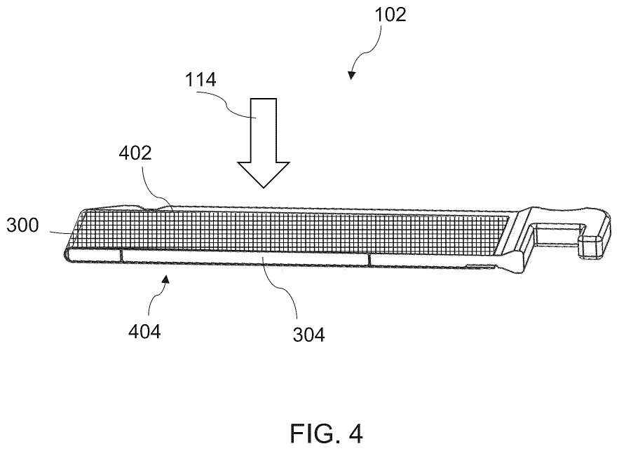

FIG. 2 is a block diagram showing components, some optional, of an inhaler device 200 configured for controlling the temperature of a source material unit 102, according to some embodiments. It should be noted that device 200 is configured to control the operational temperatures and/or heating of the upstream filter 402 and/or downstream filter 404 (which could be two different portions of the same filter, as depicted for example in FIG. 4), in order to provide the desired heating of the source material 304 in the source material unit 102. For example, in order to attain and/or retain and/or approximate a desired target temperature of the source material. In some embodiments, the target temperature is linked to the vaporization temperature of one or more substances associated with the source material 304 in the source material unit 102, such that attainment and/or maintenance and/or approximation of the target temperature allows the user 208 to inhale the vaporized substance(s).

In some embodiments, when several distinct substances are to be delivered concomitantly, and the substances optionally have different vaporization temperatures, a target temperature may be selected according to the respective vaporization temperatures, such that it is either the highest, lowest or any temperature in-between amongst the vaporization temperatures. A benefit of using the highest temperature may be faster vaporization of all substances. Using a lower temperature may result in less efficient vaporization of substances having a higher vaporization temperature but may reduce or prevent heating damage to one or more substances having a lower vaporization temperature.

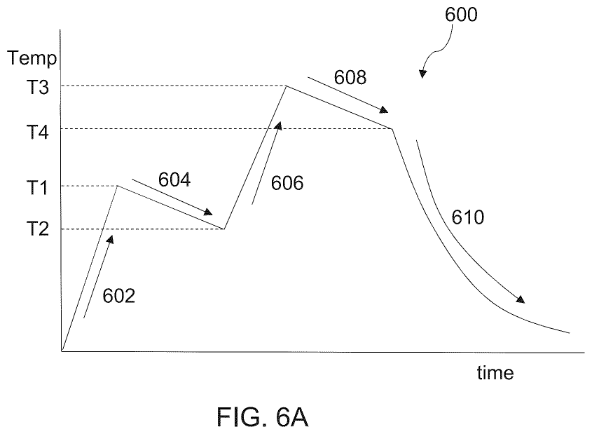

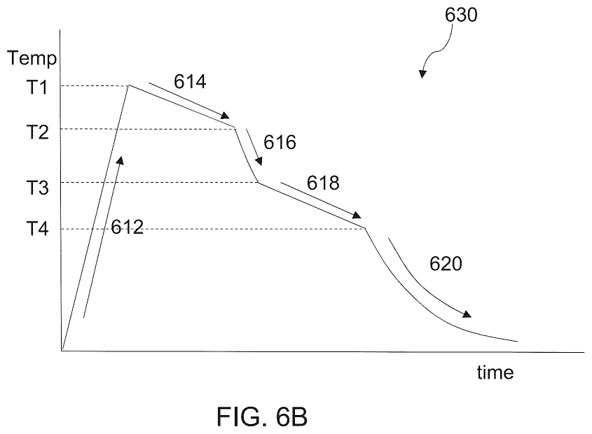

Optionally, a multi-step process 600 is performed, as depicted for example in a temperature plot shown in FIG. 6A. According to some embodiments, a first surface of substance unit is heated until a first temperature (T1) is reached (602). In some embodiments, the temperature is then reduced (604) to reach a second temperature (T2). Then, in some embodiments, subsequent heating is performed (606) to reach a third temperature (T3) being higher than the first temperature and then optionally reduced (608) to a fourth temperature (T4), after which heating is optionally terminated (610). In this example, T1 and T2 are selected according to a first target temperature, e.g. a vaporization temperature of a first substance, with T1 being optionally below a temperature capable of damaging the first substance. T3 and T4 are selected according to a second target temperature and optionally at least one of T3 and T4 is high enough to damage the substance having the lower vaporization temperature.

Optionally, a multi-step process 630 is performed, as depicted for example in a temperature plot shown in FIG. 6B. According to some embodiments, a first surface of substance unit is heated until a first temperature (T1) is reached (612). In some embodiments, the temperature is then reduced (614) to reach a second temperature (T2), Then, in some embodiments, subsequent heating is controlled (616) to reach a third temperature (T3) being lower than the first temperature. In the example shown in FIG. 6B, controlling to reach T3 (616) is depicted as a rapid cooling step (e.g. by a brief stop in heating) but in the event that T3 is a higher temperature than T2, heating may be performed. In some embodiments T3 is lower than T2 but cooling from T2 to T3 is performed while heating is maintained, for example in order to control a rate of cooling. Optionally, T2 is equal to T3 such that only the slope between T1 and T2 changes to become the slope between T3 and T4 without passing through the slope phase shown between T2 and T3. Thereafter heating is controllably reduced (618) again to a fourth temperature T4, after which heating is optionally terminated (620). In this example, T1 and T2 are selected according to a first target temperature (e.g. a vaporization temperature of a first substance, with T1 being high enough to efficiently vaporize the first and second substances; for example by being higher than the vaporization temperatures of both first and second substances), and T3 and T4 are selected according to a second target temperature being low enough to efficiently vaporize only the substance having a lower vaporization temperature (for example by being between the vaporization temperatures of the two substances).

In some embodiments, the described heating process may allow vaporizing the first substance and the second substance during the first heating period, and then terminate the release of the first substance and continue the release of only the second substance. This process may be utilized to design the release of a selected ratio of the first and the second substances according to the rate of release and/or the vaporization temperature of each of the substances.

In some embodiments, the source material unit 102 is heated from within (for example with at least one heating element or a portion thereof running through it) and an upstream surface and/or a downstream surface of the source material unit are thermally controlled, in addition to, in lieu of, or separately from heating of an upstream filter 402 and/or downstream filter 404. In some embodiments, temperature control of the upstream filter 402 and/or downstream filter 404 is effectuated by applying electrical current through at least one filter 402, 404, (whereby the filter also functions as a heating element). In some embodiments, electrical current is applied through electrodes 214, 216, which are in contact with one or both of the filters 402, 404, wherein current control is optionally regulated at least in part by temperature sensing feedback from the upstream filter 402 and/or downstream filter 404.

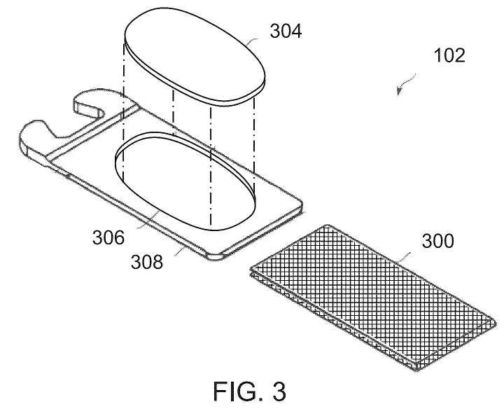

Referring to FIGS. 3 and 4, there are different operating scenarios which could be employed to provide the desired heating of the source material 304 in the source material unit 102. In some embodiments, a sloped temperature performance profile is used, optionally in combination with temperature sensing of a one of the surfaces/filters. In some embodiments, at least one sensor is disposed proximal to the upstream filter 402, for example an infrared sensor or an impedance sensor or the like for sensing temperature of the upstream filter 402. Electrical current applied to the upstream filter 402 causes it to heat to a first temperature, T1, which is, in some embodiments, higher than the target temperature. After a predetermined amount of time and/or subject to sensing an indication that a predefined temperature was reached or exceeded, the current is reduced or eliminated to instigate cooling of the upstream filter 402, optionally to a temperature, T2, being lower than the target temperature. Optionally, the target temperature is a vaporization temperature. It should be understood that, in combination with the airflow 113 through the source material unit 102, heating of the upstream filter 402 may also cause heating of the downstream filter due at least in part to convection. In some embodiments, control of the temperature of the upstream filter 402 in a sloped (e.g. hotter to cooler) profile affects the temperature of downstream filter 404 thus effectuating thermal control thereof. In some embodiments, the sloped temperature profile of the upstream and downstream surfaces actually maintains a relatively constant temperature of the source material 304 in the source material unit 102.

In a second optional example, at least one temperature sensor is disposed on each of the upstream filter and downstream filter and using the sensed temperatures of each filter, the electrical current applied to the filters is regulated to maintain each of the filters within preset windows of acceptable temperatures. That is, the controller 212 will take the sensor readings and will apply current such that the current is high enough to keep the upstream filter 402 and the downstream filter 404 within a predefined temperature range.

For example, when both upstream filter 402 and downstream filter 404 are portions of a single heating element, the current driven through the element may be controlled such that both temperatures are within the predefined range, based on combined feedback temperature sensing from both filters. Alternatively, the electric current is controlled to affect the temperature of one filter (e.g. the upstream filter) such that the temperature exhibits a predefined slope from a first temperature to a second temperature, based on sensor readings for the same filter. In another option, the electric current is delivered according to predefined parameters without real time temperature feedback or sensing.

In either scenario, more than one sensor may be used to sense temperature in either or both of the upstream and downstream surfaces/filters. In some embodiments, at least one sensor (for example, an air pressure sensor) is disposed in the inhaler device 200 for detecting airflow and/or a parameter indicative of airflow in the inhaler device 200. Optionally in either scenario, the temperature of the source material 304 may be controlled within a window, for example 10° C.-50° C. above and below a target temperature (for example a vaporization temperature of at least one substance in the source material 304). Optionally, the window is 25° C. above and below the vaporization temperature. Optionally, the window is 10° C. above and below the vaporization temperature. Optionally the window is 25° C. above and 10° C. below the vaporization temperature. Optionally the window symmetrical, with the target temperature being evenly between the first and second temperatures. Alternatively, the window is asymmetrical around the target temperature.

Optionally in addition to the upstream and downstream filter thermal control techniques and structures, such as described above, air flow within the device 200 may be controlled to work in conjunction with the filter thermal control techniques and structures. In some embodiments, flow throughout the inhaler device 200 can be generally divided into three main flow paths: a first path of flow allowing airflow 113 to pass through the source material unit 102 and exit as airflow 114 and a second optional flow of compensation airflow 112 that joins the first flow 114 to create a third main airflow 116 to the user 208 of the device. In the schematic diagram shown herein, inhalation of user 208 produces airflow 118 into the device 200. In some embodiments, the source material unit 102 is held in a use position by a holder of the inhaler device 200. The holder is configured to hold the source material unit 102 in airtight, or near airtight, communication with airflows 113 and 114 such that at least 90% of the airflow 113 passes through the source material unit 102 and the source material therein to become airflow 114 and/or that at least 95%, 97% or even at least 99% or even 100% of airflow 114 consist of airflow 113. In some embodiments, at least 98% or even 100% of the airflow 113 passes through the source material unit 102. For example, the holder may position the source material unit 102 such that only (or mostly) airflow 113 that passes through the source material unit 102 reaches a mouthpiece of the device 200 in addition only to airflow 112.

To control a rate of flow through the source material unit 102, optionally according to a target performance profile and/or to provide constant/stabilized airflow, a compensation flow regulator 106 is optionally provided to dynamically govern compensation airflow 122 into the inhaler device 200. In some embodiments, compensation airflow 122 that entered the device 200 is directed to join the flow 114 that has already passed through the source material unit 102 (via the compensation airflow regulator 106). In some embodiments, dynamic modifying of flow is performed to achieve and/or maintain a target profile of flow through the source material 304. Optionally, a target profile comprises maintaining the flow through the source material 304 at a constant rate; for example, 0.5 Liters/minute (L/min), 1 L/min, 4 L/min, or an intermediate, higher or lower rate of flow. Optionally, the profile of flow through the source material 304 comprises a varying flow profile, for example including a linearly increasing rate, linearly decreasing rate and/or any other profile.

FIG. 3 is a perspective, partially-exploded view of a source material unit 102, according to some embodiments. Optionally, source material unit 102 comprises a source material 304 (for example, a plant material), optionally formed as a pallet. Optionally, the source material is formed as a powder or other grounded material. Optionally, the source material is flattened, for example to a thickness between 0.5-1 mm, 0.05-0.5 mm, 0.2-0.8 mm, 0.5-0.9 mm or intermediate, larger or smaller thickness. A potential advantage of a flattened pallet of source material may include achieving a more unified distribution of the heat across the pallet. Another potential advantage of a flattened, thin pallet may include less interference with the flow of air passing through. Another potential advantage of a flattened, thin pallet may include a higher surface are to volume ratio which may improve vaporization, for example allowing for a higher vaporization rate.

In some embodiments, the pallet comprises a solid carrier material which is selected and/or designed to allow vaporization and inhalation of a vaporizable substance therefrom, Optionally, the vaporizable substance is applied on the pallet. Optionally applying the vaporizable substance is done by dipping in, spraying with and/or coating a carrier material with the substance. Optionally, the carrier material comprises an air-permeable matrix. Optionally, the carrier is substantially unreactive (chemically inert) with respect to the vaporizable substance when in contact therewith, at least within a temperature range as low as the lowest expected storage temperature and up-to the operational temperature (e.g. the vaporization temperature of at least one substance), possibly with some greater range of confidence (e.g. between 50° C. below a storage temperature and up-to about 50° C. above an operational temperature). Optionally, the vaporizable substance is in the form of a liquid solution. Optionally, the pallet is soaked in the solution to absorption.

In some embodiments, the source material is particulate (e.g. granulate) positioned within a cavity 306 and/or otherwise contained in a frame or other suitable structure. Optionally, source material unit 102 comprises a mechanical support for the source material 304 (for example, in cavity 306 within a housing 308, which is optionally frame shaped). Optionally, source material unit 102 comprises an attachment element for facilitating transport of the source material unit 102 (for example, latch mandibles 310). Optionally, source material unit 102 comprises means for vaporizing the source material 304 (for example, an electrically resistive heating element, optionally a filter, or a mesh, and/or a structure passing through the source material to heat the source material from within).

In some embodiments, in a constructed source material unit, source material 304 is at least partially surrounded by filter 300. The assembly of the source material and the filter holding it is supported (optionally contained) by housing 308, whereby cavity 306 of the housing allows for air to flow to and through a first side of the filter, through the source material, and through a second, opposite side of the filter.

Different examples of the above-listed elements (and components introduced in FIG. 2 are described in related applications, including U.S. Pat. Nos. 9,993,602; 10,099,020; 10,008,051; and, 9,839,241, the disclosures of which are incorporated herein by reference, as well as examples of embodiments of source material units which lack at least one of these elements. It is to be understood that the different element embodiments are optionally combined in embodiments of assembled source material units in other combinations as well (for example, any heating element design provided with any frame design). Optionally, an individual (or, optionally, a group of) used source material unit 102 is ejected after use.

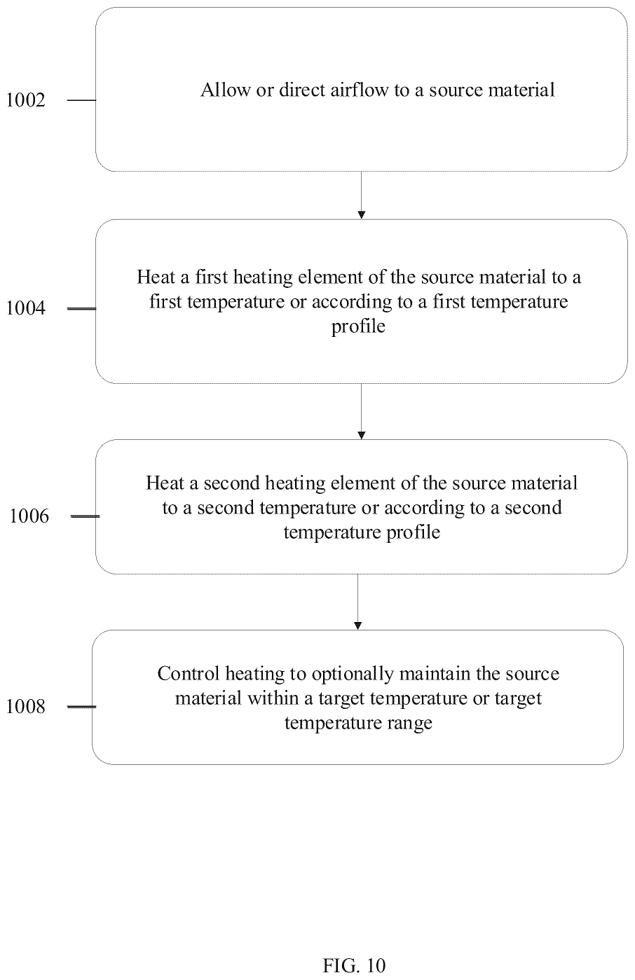

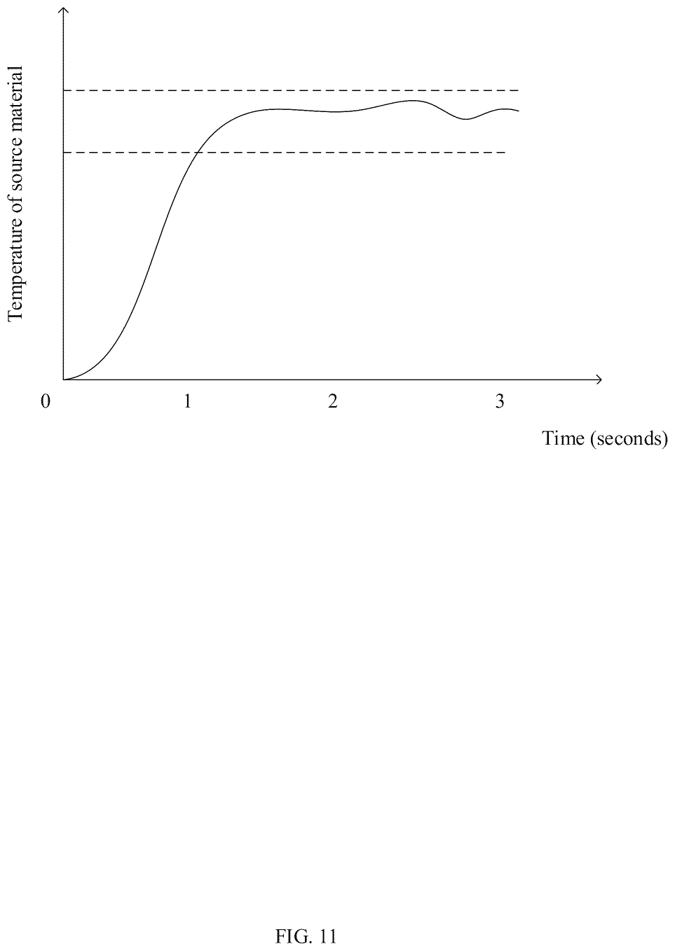

It should also be understood that a multiple source material unit structure, such as a magazine or cartridge, could be provided to any of the inhaler devices described herein such that as each individual source material unit 102 is used, a new one is supplied for use by the user from the magazine. Optionally, a used source material unit 102 remains in the source material unit structure even though it has already been used (and the entire structure is disposed of when all of the source material units therein have been used). An example for a source material unit structure is shown in FIG. 15 of U.S. Pat. No. 9,993,602 in a carousel type magazine. While a carousel is shown, the magazine could be linear (like a semi-automatic pistol magazine, except source material units 102 are fed into a usable position in an inhaler device) or of any other configuration with the objective of being able to conveniently provide the user with a plurality of source material units 102 in series or in parallel. Additional examples of source material unit structures (e.g. cartridges, magazines) are as shown for example in FIG. 10 of U.S. Pat. No. 10,099,020, showing source material units held within two separate carousels and arranged for potentially simultaneous administration; and in FIG. 11 of U.S. Pat. No. 10,099,020, which schematically illustrate a linear magazine of source material units.

In some embodiments, a plurality of source material units is pre-placed in an operable position within the inhaler device such that each of the source material units can be individually activated. Optionally, the source material units are activated serially, for example, on demand when a user's inhalation is sensed. Optionally, two or more of the source material units are activated simultaneously, for example if each source material unit contains less than a full dose or if the user desires the administration of more than a full dose in a single inhalation, and/or in order to deliver different substances from each unit.

Optionally, source material unit 102 is disposable. Potential advantages of a disposable source material unit 102 may include: containment of source material and/or substance residue for disposal; close integration of dosage support and reliable dosage transport within a vaporizer device; and/or a reduced need to maintain and/or monitor portions of the vaporizer device (such as a vaporizing heating element) which are subject to conditions that could degrade performance over time.

Optionally, the source material unit 102 is for use in a single inhalation. Potential advantages of a single-use source material unit 102 include improving the precision and/or reliability in controlling the amount of substance vaporized, reducing issues related to contamination and use damage.

In some embodiments, the source material unit 102 or source material 304 dimensions are, for example, about 6×10 mm, about 8×8 mm, about 4×6 mm or intermediate, larger or smaller dimensions across the exposed surface area. Optionally source material 304 has a thickness at a range of 0.5-1 mm, 0.2-0.8 mm, 0.5-0.9 mm or intermediate, larger or smaller thickness. Optionally, source material 304 (for example, when formed as a pallet) is positioned perpendicularly to airflow during use, such that air flows through the entire thickness of source material 304. Optionally, the thickness of the source material 304 is in the range of about 0.2-1.0 mm. Optionally, source material 304 may have a thickness greater than 1.00 mm or lesser than 0.2 mm. Optionally, the face area of the source material 304 is in the range of about 20-100 mm²; for example 20 mm², 40 mm², 50 mm², 60 mm², 80 mm², or another greater, lesser, or intermediate face area. The source material 304 is optionally formed into a square or substantially square pallet-shaped structure (for example, about 8×8×1 mm, 5×5×0.5 mm, 10×10×2 mm or intermediate, larger or smaller dimensions). Alternatively, the pallet has an oblong shape, having a length to width ratio of, for example, 1:2, 1:3, 1:4, 1:10, or another larger, smaller, or intermediate ratio of widths. In some embodiments, the pallet comprises a rectangular shape (having sharp and/or rounded corners), or any other shape, with airflow passing between the largest exposed surfaces of the material along through the shortest flow path. Optionally the airflow path through the source material corresponds to the thickness of the source material, for example being 2 mm long or less, 1 mm long or even 0.5 mm or another longer, shorter, or intermediate length. Optionally, the pallet is, for example, about 30×2×1 mm in dimension. Corresponding substance load by weight is about 10-25 mg (e.g. 13.5, 15 or 17 mg) in some embodiments. In some embodiments, the substance load of the source material 304 is selected from within a range of about 5-100 mg or 5-25 mg or 10-20 mg, or another range having the same, larger, smaller, and/or intermediate bounds.

It is a potential advantage to at least partially surround the source material 304 with a framing housing 308 for greater mechanical stability. For example, botanical substances used to form a source material 304 potentially comprise friable material, such that a source material 304 is liable to shed particles, particularly if bent and/or agitated. Enclosure within a cartridge frame allows the source material 304 to be moved within the system without applying stresses directly to the source material 304 itself and/or optionally renders it less sensitive to agitation in the event that the cartridge frame provides at least some structural support to the source material 304. In some embodiments, the overall length and width of the cartridge is about 20×10 mm, or another larger, smaller, or intermediate size. During manufacture, a framing housing is a potential advantage for formation of a pallet of the correct size for fitted occlusion of a conduit through which air flows to pick up volatiles released during heating of the pallet.

In some embodiments, vaporization of one or more substances (for example, volatile substances) associated with the source material 304 comprises heating by an electrically resistive heating element (e.g. the filter 300, optionally constructed as a mesh, or other form of resistive heating element such as described elsewhere herein). The resistive heating element optionally comprises a material which displays substantial electrically resistive heating; for example, nichrome (typical resistivity of about 1-1.5 μΩ·m), FeCrAl (typical resistivity of about 1.45 μΩ·m), stainless steel (typical resistivity of about 10-100 μΩ·m), tungsten (typical resistivity of about 52.8 nΩ·m), and/or cupronickel (typical resistivity of about 19-50 μΩ·m). According to the choice of metal, parameters such as heating element length and width, metal thickness, aperture size and/or aperture pattern are adjusted to comprise a total resistance across the resistive heating element which is, for example, in the range from about 0.05-1Ω, 0.5-2Ω, 0.1-3Ω, 2-4Ω, or within another range having the same, higher, lower, and/or intermediate bounds.

FIG. 4 is a cross-sectional view of a source material unit 102, according to some embodiments. In some embodiments, the source material 304 which is embedded in the source material unit 102 has a first, upstream surface, being filter 402 and a second, downstream surface, being filter 404 (which is on the obverse side of the source material unit 102 relative to the first upstream surface 402). During operation and/or use of the inhaler, airflow passes through the transverse distance between the surfaces, within which source material 304 is disposed. In some embodiments, these surfaces comprise or are formed of filters 402, 404. Optionally, filters 402 and 404 are part of a single filter 300, which is generally U-shaped and folded over the source material unit 102 such that one side of the filter is disposed upstream of the source material and the opposite side of the filter is disposed downstream of the source material. In some embodiments, the upstream filter and the downstream filter are separate units. In some embodiments, the upstream surface and downstream surface are not filters, but the surfaces of the source material 304 itself.

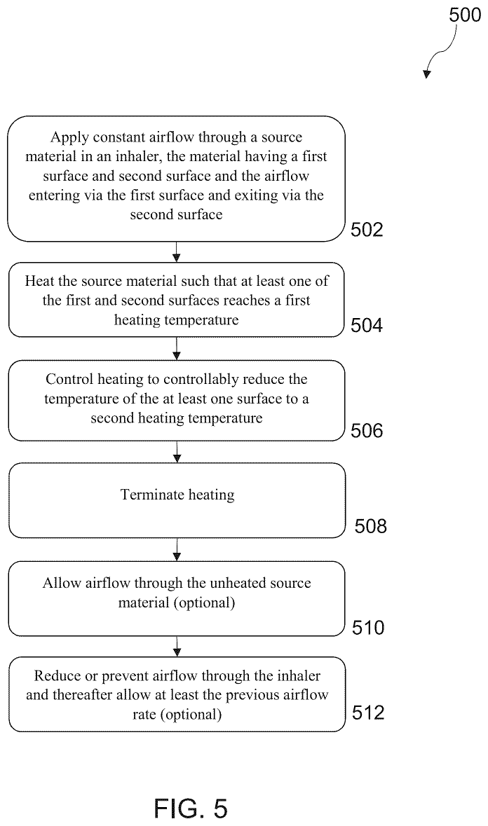

FIG. 5 is a flowchart 500 of a method for controlling the temperature of a source material unit 102 in an inhaler device 200, according to some embodiments. In some embodiments, once user 208 inhales, the inhaler device 200 modifies airflow (optionally) to apply (502) constant airflow through the source material 304 having an upstream surface (or filter) and a downstream surface (or filter). Heating (504) is applied, directly and/or indirectly, to the source material 304 such that surface 402 and/or surface 404 reaches a first temperature. For example, heating (504) is applied by heating the upstream surface 402 to effectuate heating of the source material 304 through conduction. In some embodiments, heat is conducted from one or both of the surfaces directly to the source material. In some embodiments, heat is conducted across the two surface and/or across a portion connecting the surfaces, for example, in the U-shape configuration. Optionally one or both surfaces 402, 404 are heated by driving an electric current through one or more heating elements being in contact with one or both surfaces. Optionally the one or more heating elements comprises the filter or a portion thereof. In some embodiments downstream surface 404 may be cooled or heated through convection (airflow 114 passing through heated source material 304). Optionally, upstream surface 402 may be cooled through convection by airflow 114 passing into source material 304.

In some embodiments, once heating (504) is accomplished and the first temperature is reached, it is controlled (506) to reduce the temperature of the surface 402, 404 to a second temperature. As described elsewhere herein, the transition from the first temperature to the second temperature creates a sloped temperature profile for at least one of the surfaces 402, 404 and optionally a relatively uniform temperature profile for at least a portion of the source material 304 in the source material unit 102 in between the surfaces 402, 404.

In some embodiments it may be desired to reach a non-uniform (i.e. varying) temperature distribution across the source material (such as across the thickness of a pallet of source material and/or across a surface of the pallet). Optionally, in such situation, a temperature profile of the upstream and/or downstream surface may be selected in accordance with the desired temperature distribution across the source material.

In some embodiments, the downstream surface 404 is heated (504) to a first temperature by directly applying electrical current to the downstream surface 404 (i.e. the downstream surface 404 is sensed and current is applied by the controller 212 to directly control the temperature of the surface 404, which is distinguished from sensing the temperature of the upstream surface 402 and controlling the temperature of the upstream surface 402 to indirectly control the temperature of the downstream surface 404 through convection and/or conduction).

In some embodiments, after a period of time at the first temperature and/or a period of time transitioning to a second temperature and/or a period of time at the second temperature, heating is terminated (508). A specific example is described with more detail below. This period of time may be proportionate to an amount of the substance that is to be delivered to the user during a given inhalation.

Optional actions include allowing (510) airflow through the source material 304 after heating has been terminated (508), for example to clear source material residue from the inhaler device 200, and reducing or preventing airflow (512) through the inhaler device 200, for example to facilitate cooling of the source material unit 102/source material 304/upstream surface 402/downstream surface 404. In some embodiments, valve 108 closing takes up to 100-500 milliseconds (ms). Optionally, valve 108 closing takes up to 150-400 ms. Optionally, valve closing takes up to 200-300 ms. Optionally, valve closing less than 100 ms. In some embodiments, the valve 108 remains closed for up to 1 second. Optionally, the valve 108 remains closed for up to 850 ms. Optionally, the valve 108 remains closed for up to 700 ms. In some embodiments, airflows within a range of 0.5 L/m to 3 L/m through the source material 304. Optionally, air flows within a range of 0.8 L/m to 2 L/m.

In some embodiments, for example when using cannabis as the source material 304, once the user 208 begins inhalation, the inhaler device compensates for insufficient or excess airflow, for example using the compensation flow regulator 106 and its valve 108, to stabilize airflow 114 through at least the source material unit 102 containing the cannabis. In some embodiments, inhalation is sensed by the pressure sensor 110, for example by sensing a pressure drop in the inhaler device (e.g. a drop of at least 50 Pa). In some embodiments, “stabilized airflow” means that the airflow is within a predefined set of parameters, including range, set point and/or over a duration of time, for example (−300)-(−400) Pa set point, to ±35 Pa and for at least 150 ms. For safety and/or quality control reasons, if stabilization is not achieved within a certain timeframe (as can be set at the controller 212 through the user interface 201 or physician interface 203 or be factory pre-programmed), for example 700 ms, operation of the inhaler device 200 is terminated and the user 208 is alerted.

Once airflow stabilization is achieved, heating of the source material unit 102 is activated to achieve a first temperature of at least the upstream surface as sensed, T1, of 200° C. for 400 ms with the objective being to heat substances including cannabinoids, and particularly including at least one of THCA and THC within the cannabis source material to its vaporization temperature and optionally to cause carboxylation thereof. Heating is then controlled to allow cooling of the upstream surface to 165° C. at the end of about 1220 ms (for delivery of 0.5 mg of THC in a source material 304 containing about 3 mg THC and THCA within about 13.5 mg cannabis granulate) after which time the heating is terminated.

In some embodiments, if temperatures do not rise to intended levels and/or if the temperature exceeds the intended levels, the process can be terminated by the controller 212. Optionally, the user is notified. Optionally, user 208 is provided with the ability to terminate the process at any time, for example through the user interface 201.

In some embodiments, heating is stopped if a heating profile and/or if a target temperature is deviated from by a predetermined temperature value or percentage and/or a predetermined time at temperature. For example, the predetermined temperature value is at least 3° C. higher or lower than the selected temperature. Optionally, the predetermined temperature value is at least 5° C. higher or lower than the selected temperature or even at least 7° C., 10° C. or 15° C. higher or lower than the selected temperature. In some embodiments, a temperature is deemed deviate from a selected temperature if the deviation lasts a period of time being at least 1% of the length of the period of temperature reduction. Optionally, a temperature is deemed deviate from a selected temperature if the deviation lasts a period of time being at least 2%, at least 4% at least 5% or even at least 10% of the length of the period of temperature reduction. In some embodiments, a temperature is deemed to deviate from a selected temperature if the deviation lasts a period of time being at least 15 ms long. Optionally, a temperature is deemed to deviate from a selected temperature if the deviation lasts a period of time being at least 10 ms, 15 ms, or even 25 ms long.

Additionally or alternatively, heating is stopped in the event of a deviation from a selected airflow and/or air pressure parameter by at least a predetermined airflow and/or pressure value or a measured value indicative thereof. In some embodiments, the predetermined pressure value is at least 5 Pa, at least 10 Pa, at least 15 Pa, at least 25 Pa, or even at least 35 Pa higher than the selected air pressure parameter. In some embodiments, the predetermined pressure value is at least 5 Pa, at least 10 Pa, at least 15 Pa, at least 25 Pa, or even at least 35 Pa lower than the selected air pressure parameter. Optionally, an airflow parameter is deemed deviate from a selected airflow parameter if the deviation lasts a period of time being at least 5% of the length of the period of temperature reduction. Optionally, the air pressure parameter is deemed deviate from a selected the air pressure parameter if the deviation lasts a period of time being at least at least 2%, at least 5% or even 10% of the length of the period of temperature reduction. In some embodiments, the air pressure parameter is deemed to deviate from a selected air pressure parameter if the deviation lasts a period of time being at least 5 ms long. Optionally, the air pressure parameter is deemed to deviate from a selected air pressure parameter if the deviation lasts a period of time being at least 25 ms long, at least 35 ms, at least 50 ms or even at least 70 ms long.

In some embodiments, airflow through the source material and/or the flow path leading to an inhaling user's mount continues after heating is terminated in order to flush or clear residue from the inhaler device 200 and/or to facilitate cooling of the source material unit.

In some embodiments, the process from inhalation commencement of the user 208 to the end of the pulsing is no longer than about 3 seconds, no longer than about 5 seconds, no longer than about 1.5 seconds or intermediate, longer or shorter duration.

It should be understood that the temperatures, times, pressures (collectively a performance profile) change depending on various factors such as the source material or materials being used, the amount of source material(s) being used, the thickness of the source material(s) and/or the source material unit, and the like. Particularly since different materials exhibit different vaporization temperatures.

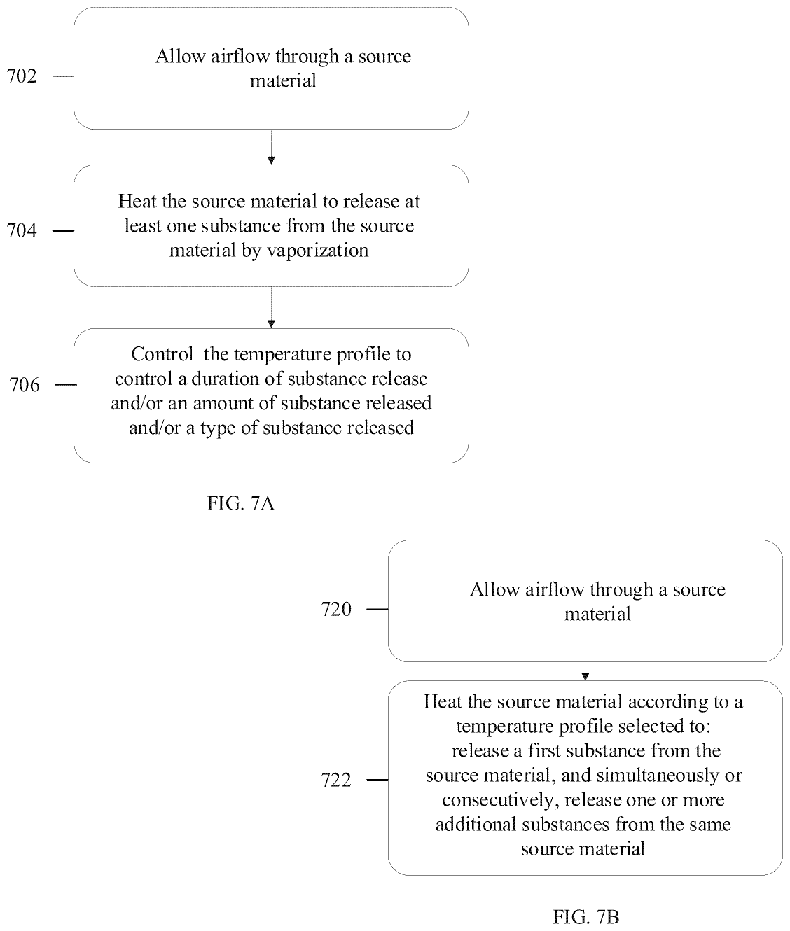

FIGS. 7A-B are flowcharts of methods for selecting a temperature profile to control or affect release of one or more substances, according to some embodiments.

Referring to FIG. 7A, in some embodiments, airflow is allowed through a source material (702), for example through source material held or supported by an air-permeable frame. Optionally, airflow is directed through the source material, for example via a conduit which is in fluid communication with the source material unit. At 704, in accordance with some embodiments, the source material is heated to release at least one substance from the source material by vaporization. At 706, in accordance with some embodiments, a temperature profile of heating the source material is controlled to control and/or affect one or more of: a duration of substance release, an amount of substance released, and optionally a type of substance released (if the source material contains more than one releasable substance).

In some embodiments, two or more different substances are released from the same source material (for example THC, CBD released from cannabis.

In some embodiments, one substance is a chemical derivative of another substance, for example, THC and THCA, CBD and CBDA.

In some embodiments, two or more different substances are released from two or more types of source materials, optionally contained within the same unit or frame.

In some embodiments, the temperature profile is controlled based on the vaporization temperature of each of the substances being released. Optionally, a temperature value and/or a trend in the temperature change (e.g. rise, drop) controls or affects a time in which the substance is released; a duration along which the substance is released; an amount of substance released. By controlling the heating profile in accordance with the thermal and/or chemical properties of the source material and/or of the substance(s) released from it, a desired combination of substances may be released, including selected ratios and/or relative timing of release of the substances.

FIG. 7B relates to timing of substance release by controlling the temperature profile, according to some embodiments. At 720, airflow is allowed (and/or directed) through a source material, in accordance with some embodiments. At 722, in some embodiments, the source material is heated according to a temperature profile selected to release a first substance and, simultaneously or consecutively, release one or more additional substances from the same source material. In some embodiments, there is in overlap between releasing of a first substance and releasing of one or more additional substances. Additionally or alternatively, substances are released one after the other, optionally with a time interval in between.

In some embodiments, the passing of airflow (e.g. the airflow rate, volume) through the source material is controlled, optionally in a synchronous manner to the heating profile, to control substance release. In an example, increasing the airflow rate (for example once a selected heating temperature had been reached) may accelerate release of a first substance while having a reduced or lower effect on releasing a second substance.

A potential advantage of controlling release of more than one substance by controlling the heating profile and/or by controlling the airflow profile through the source material may include improving the accuracy of substance release, for example providing for improved control over a timing of release, the amount of substance released, the type of substance released. This dual control (of the airflow profile and of the heating profile) may provide a set of multiple control parameters (e.g. airflow rate, airflow volume, heating rate, maximal heating temperature, minimal heating temperature, heating gradient, duration of heating, duration of airflow and/or other control parameters), where variation in one or more of the parameters may generate a controlled change the content of substances being released (types, durations, amounts, ratios, etc).

The table below lists some examples of plant materials, one or more active ingredients releasable from the plant material(s), a melting point of the active ingredient and a boiling point of the active ingredient. The melting point may refer to a temperature in which an ingredient is transferred form a solid state to a liquid state; the boiling point may refer to a temperature in which the ingredient vaporizes.

In some embodiments, heating is applied to raise a temperature of the source material to a temperature that is between the melting point and the boiling point. In some embodiments, this target temperature is selected as a tradeoff between a temperature which is too low as compared to the boiling point, potentially increasing the time required for release of the ingredient; and a temperature which is too high, for example, the boiling point itself or above it, which may result in an overshoot in the amount of ingredient released (for example, a large amount released over a too short time period).

In some embodiments, the target temperature is selected taking into account that different molecules (even of the same ingredient) reach the boiling point at different time points, and not necessarily altogether. Some molecules may vaporize before the source material temperature reaches the target temperature.

| | | Melting point | Boiling point |
|---|---|---|---|
| | Active | (° C., at | (° C., at |
| Botanical Name | ingredient | 760 mmHg) | 760 mmHg) |
| Papaver somniferum | Morphine | 254 | 476.2 ± 45.0 |
| (opium poppy) | Codeine | 155 | 250 (at 22 mm/Hg) |
| | Thebain | 183 | 467.6 ± 45.0 |
| | Oripavine | 200-201 | 480.5 ± 45.0 |
| San Pedro (Echinopsis | Mescaline | 35.5 | 312.1 ± 37 |
| pachanoi) | Hordenine | 117-118 | 270.2 ± 23.0 |
| | Tyramine | 160-162 | 325.2 ± 0.0 |
| Kratom (Mitragyna | Mitragynine | 92-95 | 560.3 ± 50 |
| speciosa) Thom, | Mitraphylline | 222.57 | 555.2 ± 50.0 |
| Thang, and Biak | mitragynine | | 580.9 ± 50.0 |
| | pseudoindoxyl | | |
| | Rhynchophylline | | 560.8 ± 50.0 |
| Catha edulis (Khat) | Cathinone | 46.81 | 255.0 ± 23.0 |
| | Cathine | 49.1 | 288.1 |
| Sceletium tortuosum | Mesembrine | 147.91 | 419.2 ± 45.0 |
| (Kanna, aka, | Mesembrenone | 148.77 | 383.9 ± 42.0 |
| channa, kougoed) | | | |
| Psilocybin mushroom | Psilocybin | 224 | 523.4 ± 60.0 |
| | Psilocin | 174.5 | 392.8 ± 32.0 |
| Amanita Muscaria | Muscimol | 146 | 325.0 ± 27.0 |
| | Ibotenic acid | 294.61 | 458.8 ± 45.0 |

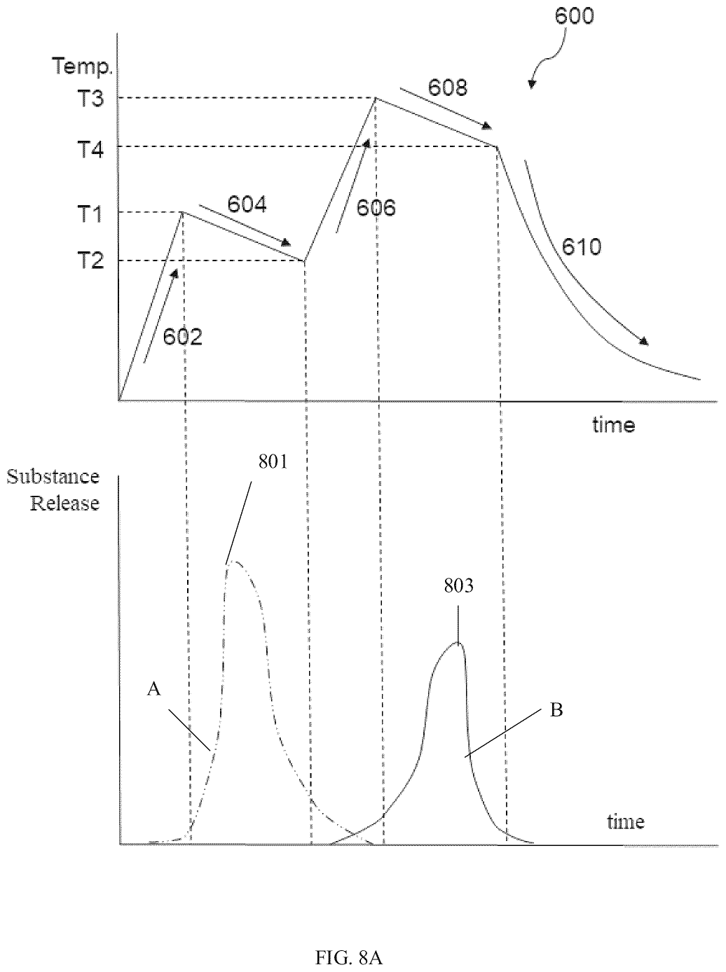

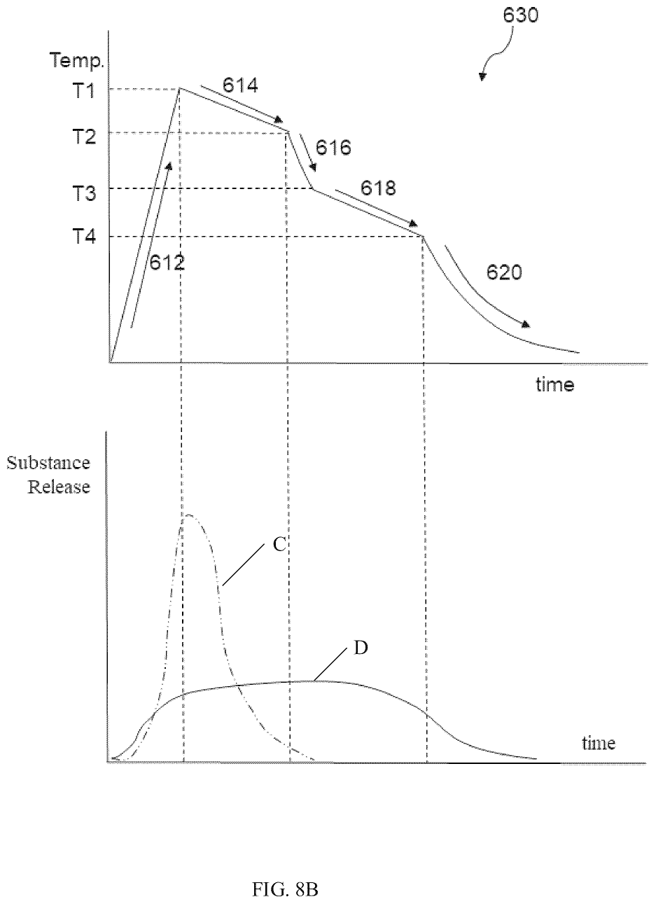

FIGS. 8A-B graphically show examples of substance release in correlation with temperature profiles for example as shown in FIGS. 6A-B, according to some embodiments.

In FIG. 8A, heating to temperature T1 is shown to generate release of a first substance “A”, indicated by the dashed line. In some embodiments, the amount of substance released reaches a peak amount 801 at a certain time period following reaching T1, for example, between 1 msec-2seconds, between 0.5 seconds-3 seconds, between 0.1 seconds-1 seconds or intermediate, longer or shorter time periods following reaching T1. Optionally, reducing the heating to reach T2 from T1 gradually reduces the amount of substance A being released, optionally to a complete stop. Additionally or alternatively, substance A at this point in time had been fully released (such that no additional substance A can be released from the source material), causing the reduction and/or stopping of the release.

In some embodiments, release of substance “B” (indicated by the continuous line) begins while substance A is still being released, as shown. Alternatively, release of substance B begins only after release of substance A has stopped (e.g. immediately after or after a certain time period from when release of substance A has stopped). A peak amount 803 of substance B is reached, in this example, a certain time period after heating again to reach temperature T3. Optionally, reducing (or stopping) the heating (reaching T4 or a lower temperature) slows down release of substance B. Additionally or alternatively, substance B at this point in time had been fully released (such that no additional substance A can be released from the source material), causing the reduction and/or stopping of the release.

In some embodiments, as shown in this example, temperature T1 is selected to be high enough to generate release of substance A (optionally being equal to or higher than a vaporization temperature of substance A, optionally being within the range of 5° C., 2° C., 10° C. or intermediate, larger or smaller range of the vaporization temperature). In some embodiments, temperature T1 is selected to be low enough so as to reduce or prevent release of substance B, for example in the event substance B has a higher vaporization temperature than substance A. Optionally, as shown, substance B is released only when heating to a higher temperature T3 (higher than T1). Optionally, upon releasing of substance B, raising the temperature from T2 to T3 does not result in release of substance A because a full potential amount of substance A was already released.

Additionally or alternatively, in some embodiments, releasing (or preventing/reducing release) of a substance is achieved by intentionally causing one or more molecular changes to the substance. Some examples of molecular changes include deoxidization, deterioration, hydrolysis and/or other molecular changes. Optionally, a change in molecular structure affects a vaporization temperature of the substance.

In the example of FIG. 8B, a temperature profile is selected to generate fast release of a relatively high amount of a substance “C” (indicated by the dashed line), optionally simultaneously or at least partially overlapping with slow release of a relatively low, constant amount of a substance “D” (indicated by the continuous line). In this example, heating to a temperature T1 causes immediate release of a relatively high amount of substance C. Simultaneously, substance D is released at a lower rate and/or amount. When the heating is gradually reduced, release of substance C stops closely after reaching T2, while release of substance C continuous in a relatively constant manner until terminating heating, following T4.

While the examples of FIGS. 8A-B schematically show release of two substances, it is noted that more substances (e.g. 3, 4, 6, 10, 20) or intermediate, larger or smaller number of different substances may be released. Optionally, two or more substances are released during a single user inhalation.

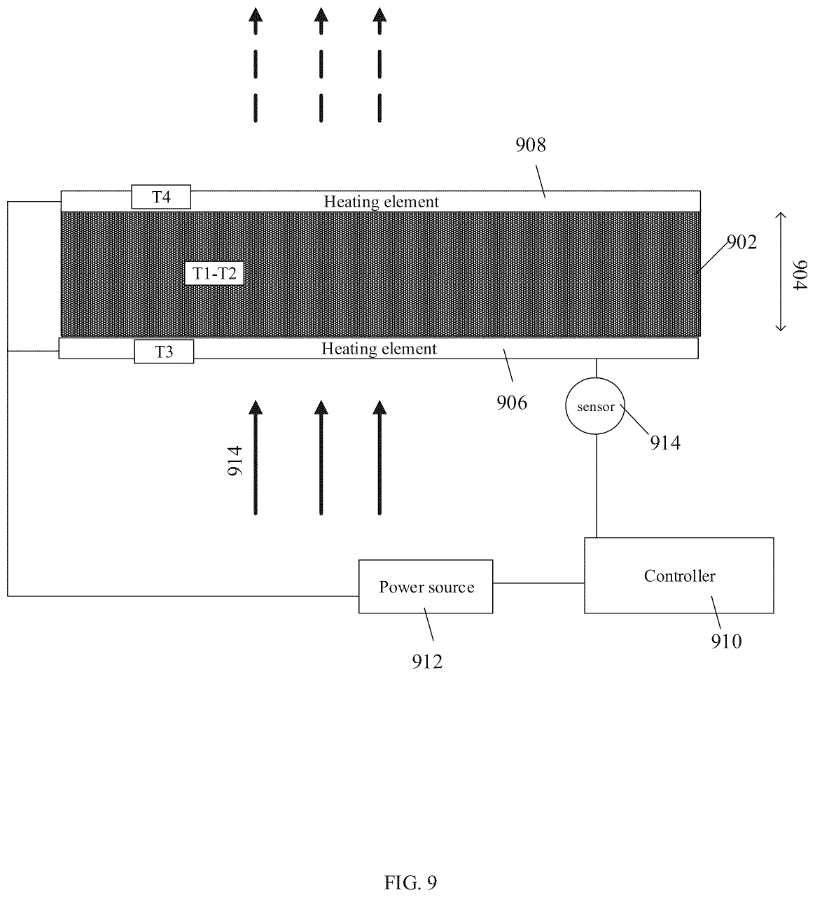

FIG. 9 is a schematic diagram of a heating module for heating a source material, according to some embodiments. A module for example as described herein may be implemented in an inhaler device for delivery of one or more substances released from a source material to an inhaling user.

In some embodiments, source material 902 is packaged in the form a pallet. In some embodiments, pallet comprises a thickness 904 of between 0.5-1 mm, 0.05-0.5 mm, 0.2-0.8 mm, 0.5-0.9 mm or intermediate, larger or smaller thickness. In some embodiments, the pallet comprises particles, optionally ground and/or otherwise processed particles. In some embodiments, the source material includes or is formed of plant matter which maintained its microstructure botanical structure intact. In an example, the source material comprises cannabis trichomes.

In some embodiments, the particles are dispersed with spaces therebetween such that air is allowed to flow through the source material, optionally passing in between particles.

In some embodiments, the source material pallet is heated by one or more heating elements. In some embodiments, as shown in this example, two heating elements 906, 908 are positioned to heat the pallet from two opposite directions. Optionally, each heating element is in contact with a surface of the pallet, for example, extending across at least a portion of the surface of the pallet.

In some embodiments, the heating element comprises an electrically resistive element, being configured to heat when electrical current is applied, for example applied via an electrode which contacts or is moved into contact with the heating element. In some embodiments, the heating element is shaped to allow for air to flow through, for example including spaces or openings. In some examples, the heating element is formed as a mesh, for example a stainless steel mesh.

In some embodiments, a controller 910 is configured to control one or more parameters of heating the heating element(s), for example: initiation of heating, a duration of heating, termination of heating, increasing of heating, reducing of heating, setting a target heating temperature of the heating element(s), setting a target heating temperature and/or a target temperature range for the source material itself, and/or other heating parameters.

In some embodiments, powering for actuating heating of the heating elements (such as by conducting electrical current to the heating elements) is supplied by a power source 912. In some embodiments, controller 910 controls the power supply by power source 912.

In some embodiments, a sensor 914 is positioned and configured for measuring a temperature of at least one of: heating element 906, heating element 908, the source material 902 or certain portions thereof. In some embodiments, a plurality of sensors (e.g. 2, 3, 5, 6, or intermediate, larger or smaller amount of sensors) are used, optionally located at different locations. Sensor 914 may be placed at or adjacent the heating element, disposed inside the pallet, disposed on the surface of the pallet, and/or other locations suitable for measuring a temperature of one or both of the heating elements and/or of the source material. In some embodiments, sensor 914 measures the temperature of the heating element by contacting the heating element. In some embodiments, sensor 914 is measures the temperature of the source material surface by contacting the surface. Additionally or alternatively, sensor 914 is configured to measure a temperature of the heating element and/or of the source material surface from a distance, for example, from a distance of 0.1-10 mm from the heating element or from the source material surface. For example, an IR sensor is positioned at a distance of 3 mm-20 mm, 6 mm-15 mm or intermediate, larger or smaller distance from the heating element for detecting a temperature of the heating element.

In some embodiments, sensor 914 is an impedance based temperature sensor, a light based temperature sensor, a resistance based temperature sensor, an infrared temperature sensor.

Additionally or alternatively, a resistance of the heating element is detected (e.g. via suitable circuitry) and used as a measure of temperature.

In some embodiments, controller 910 controls heating according to an indication received from the sensor. Optionally, closed-loop temperature control is performed, where, for example, the controller initiates heating of the heating element(s); a temperature of one or both of the heating elements and/or of the source material is detected by the sensor; an indication of temperature is received by the controller; the controller sets further heating or instructs to stop heating based on the indication from the sensor. In some embodiments, the sensor measures the temperature periodically, for example, at selected times before/during and/or following heating and/or at certain time intervals. Optionally, the sensor continuously tracks the temperature.

In some embodiments, controller 910 sets heating of one or both of the heating elements to a temperature suitable to cause the source material to be heated to a temperature range in which one or more substances are vaporized from the source material. In some embodiments, heating element 906 and/or heating element 908 are each heated to a temperature different than a target vaporization temperature (or temperature range) of the source material. Optionally, the heating elements are heated to a different temperature from each other.

For example, for heating the source material to a temperature range having a low threshold at T1 and a high threshold at T2, a heating element may be heated to a third temperature T3. Optionally, T3 is higher than T2. Optionally, the second heating element is heated to a fourth temperature, T4, being higher or lower than T3.

In an example, for releasing THC from cannabis, the source material is heated to a temperature 150° C. within a range of +/−15° C., +/−20° C., +/−30° C. or intermediate, higher or lower. Optionally, the heating element is heated to a temperature higher than 150° C., for example, 170° C., 180° C., 200° C., 210° C., 220° C. or intermediate, higher or lower temperature.

In another example, for releasing CBD from cannabis, the source material is heated to a temperature 160° C. within a range of +/−15° C. range of +/−15° C., +/−20° C., +/−30° C. or intermediate, higher or lower.

In some embodiments, a temperature to which a heating element is heated is selected to cause at least 75%, at least 80%, at least 85%, at least 90%, at least 95% or intermediate, smaller or larger percentage of the source material to be heated to the vaporization temperature range.

In some embodiments a temperature to which a heating element is heated is selected to maintain at least 75%, at least 80%, at least 85%, at least 90%, at least 95%, at least 99%, or intermediate, smaller or larger percentage of the source material below a combustion temperature of the source material.

In some embodiments a temperature to which a heating element is heated is selected to maintain at least 75%, at least 80%, at least 85%, at least 90%, at least 95%, at least 99%, or intermediate, smaller or larger percentage of the source material below a maximal temperature threshold, for example to prevent release of one or more substances which vaporize at a higher temperature than one or more of the substances selected for vaporization.

In some embodiments, a heating profile of one or both of the heating elements is selected to cause the source material to be heated to a certain temperature, temperature range, or temperature profile. A temperature profile may vary in time and/or in space. For example, heating may be controlled to obtain a selected temperature distribution along the thickness of the pallet, across the surface(s) of the pallet, and the like. For example, heating may be controlled to obtain a selected temperature profile over time. An example may include heating the source material to a peak temperature, maintaining the source material within a selected temperature range optionally for a selected period of time (e.g. throughout an inhalation of a user), then optionally terminating heating.

In some embodiments, the controller is programmed to set heating parameters (e.g. target temperatures or ranges, duration of heating, initiating and/or terminating of heating) based on properties of the source material, for example: the type of source material, the thickness of the pallet, the surface area of the pallet, the density of the source material particles, the packing configuration of the source material; the size of the source material particle (e.g. diameter); the amount of source material.

In some embodiments, the controller is programmed to set heating parameters based on properties of airflow which carries the released substance(s) from the source material. In some embodiments, heating of the source material is affected by flow of air through and/or across the source material. For example, a temperature distribution along the pallet (e.g. along the thickness dimension) is affected by the direction and/or rate and/or volume of air passing through. In an example, air flows through the pallet in a direction indicated by arrows 914. If both heating elements are heated to a similar temperature (e.g. T3=T4), the passing of airflow may cause layers closer to the downstream heating element 908 to be heated to a higher temperature than layers located at a more upstream direction, towards element 906. In some cases, this may be a result of convection of heat by the airflow and/or conduction of heat by the source material. Therefore, in some embodiments, the controller is pre-programmed with and/or is configured to calculate the temperature profiles required for bringing the source material to a desired vaporization temperature range, taking into account parameters of the airflow.

In some embodiments, heating element 906, in addition to heating the source material, heats the air flowing into the source material. A further effect of the airflow may include cooling of source material portions, for example portions located at the entry of airflow into the pallet.

In some embodiments, the controller is programmed to set heating parameters based on the physical properties of the pallet and/or surrounding structures, for example, based on a contact surface area of a heating element with the pallet; based on a distance between the heating elements; based on a distance between a heating element and the pallet, if such exists; based on the shape of a frame and/or other supporting structure in which the pallet is received or held, based on the electrical resistance/conductance properties of the material forming the heating element (e.g. the mesh), and the like.

In some embodiments, the opposing heating elements are formed as a single piece, for example having a “U” shape, with the arms of the “U” extending across the surfaces of the pallet. In some embodiments, the “U” shape connects the two planar portions that define the opposing heating elements. In some cases, during use, the bending portion of the “U” shape is heated most (optionally due to heat conduction from the two planar portions, due to heat convection, due to lack of airflow therethrough and/or other causes). In some embodiments, to reduce or prevent an effect of a potentially overheated bending portion of the “U”, contact between the bending portion and the source material is reduced or prevented, for example by a spacer (e.g. a part of the frame holding the pallet is positioned intermediate the bending portion and the pallet).

In some embodiments, electricity is applied to the U shaped element as a single unit. Optionally, current is conducted evenly to both arms of the U shape (such as to and through meshes forming the arms). In some cases, due to the direction of airflow passing through the pallet and the heating elements, one arm of the “U” is heated more than the other. Optionally, by sensing a temperature of only arm of the “U”, a temperature of the opposite arm can be calculated or estimated. In some embodiments, control of heating takes into account this pre-known difference in the actual temperatures of the heating elements on both arms of the “U”.

FIG. 10 is a flowchart of a method for controlled heating of source material in in accordance with some embodiments.

In some embodiments, for releasing one or more substances from a source material by vaporization, airflow is passed to and in some embodiments through the source material before and/or during and/or following heating of the source material, for example immediately following heating.

In some embodiments, airflow is allowed and/or directed to the source material (1002). Optionally, airflow passes through the source material, for example entering on one side of the pallet and exiting from an opposite side of the pallet (for example flowing along the thickness dimension of the pallet). Additionally or alternatively, air flows across one or more surfaces of the pallet. Optionally, one or more surfaces of the pallet are at least partially exposed so as to allow for the flow of air to pick up vapors of the released substance.

In some embodiments, a first heating element of the source material is heated to a first temperature or according to a first temperature profile (1004). Optionally, the heating element is heated until reaching a selected temperature, which, in some embodiments, may be further maintained constant over time. Optionally, the heating element is heated according to a varying temperature profile, which includes, for example, a plurality of temperatures to be reached at certain timings.

In some embodiments, the heating element is heated to a temperature which is different than a target temperature for the source material and/or different (i.e. does not fall within) a target temperature range for the source material. In some embodiments, the target temperature or target temperature range for the source material include a temperature or range in which one or more selected substances vaporize from the source material.

In some embodiments, two or more heating elements of the source material are heated. At 1006, in accordance with some embodiments, a second heating element of the source material is heated to a second temperature or according to second temperature profile. In some embodiments, the second temperature or second temperature profile are different than the temperature or profile according to which the first heating element was heated. For example, one heating element is heated to a temperature that is at least 20%, at least 40%, at least 60%, at least 80% higher than the other heating element. For example, one heating element is heated to a temperature that is at least 5° C., at least 10° C., at least 20° C., at least 40° C., at least 50° C., at least 70° C., at least 100° C. or intermediate, higher or lower temperature higher than the other heating element. For example, one heating element is heated by increasing the heating and then stopping, while the other heating element is continuously heated. For example, one heating element is heated before the other.

Optionally, heating of the two or more elements is synchronized or correlated to precisely heat the source material to the target temperature or range. For example, timing of heating of the two or more heating elements is set; a temperature profile for each of the heating elements is set, where, as previously described herein, the temperature profile may be different for each of the heating elements.

In some embodiments, heating is controlled, optionally to maintain the source material within the target temperature or target temperature range (1008). In some embodiments, the target temperature or range are maintained for a duration selected according to the amount of substance to be released and taking into account the rate of substance release. In some embodiments, the target temperature or range are maintained for a duration which is as long as an inhalation of the user. In some embodiments, the target temperature or range are maintained for as long as needed to release all potential substance from the source material.

In some embodiments, heating is controlled to ensure that substantially all portions of the pallet are heated to the target temperature or range. Optionally, heating is controlled to ensure that no portions of the substance heat to a temperature beyond a defined maximal threshold, for example to prevent or reduce release of a substance which vaporizes at a higher temperature and/or to prevent or reduce combustion of the source material or its components.

In some embodiments, control of heating is carried out with the use of one or more sensors which provide indications related to the temperature of the heating element(s), temperature of the source material or portions of it, temperature of the pallet surrounding, flow rate, vaporization rate, and/or other indications.

In some embodiments, controlling heating comprises increasing and/or reducing an amount of energy inputted for heating the heating element. Optionally, power supply to the heating element is modified. Optionally, an electrical current applied to the heating element (e.g. via an electrode) is modified.

In some embodiments, a system for example as described herein (for example, a system controller) automatically sets parameters of heating. In some embodiments, parameters are set according to a look-up table, which in some examples ties between parameters such as: an airflow rate through the pallet, a thickness of the pallet, a density of the source material being used, and/or other parameters with the temperature profiles for heating the one or more heating elements.

In some embodiments, heating is modified based on the look-up table. For example, in response to a change in the rate of airflow through the pallet, the controller may modify the heating profile of the heating element(s), e.g. by reducing or increasing a temperature of the heating element.

FIG. 11 is a graphical representation of a temperature profile of the source material over time, according to some embodiments.

In the example shown, upon initiation of heating at 0 seconds, the source material is heated (optionally in a linear or close to linear manner) to reach the target temperature or target temperature range, being the range indicated between the dashed lines.

In some embodiments, heating is from a room temperature or an ambient temperature, for example 25° C., 20° C., 18° C. or intermediate higher or lower temperature.

In some embodiments, the source material is rapidly heated to the target temperature or range, for example within a time period of less than 1.5 seconds, less than 1 second, less than 0.8 seconds, less than 0.5 second or intermediate, longer or shorter time periods. In some embodiments, the source material is heated to a target temperature (optionally vaporization temperature) within 200-600 milliseconds of heating, for example, 300, 400, 500 milliseconds of heating).

In some embodiments, at the next stage of heating, when the source material has reached the target temperature or range, heating is controlled to maintain the source material within the target range. In some embodiments, heating may be applied to maintain the source material within the target temperature range for a time period of between 0.5 seconds-10 seconds, for example 2 seconds, 4 seconds, 6 seconds or intermediate, longer or shorter time periods. In the example shown, heating is controlled to maintain the source material within the target range for a time period of about 2 seconds (between second 1 and second 3). Optionally, the time period during which the temperature is maintained at the target range is as long as a single inhalation of the user from the device.

In some embodiments, a duration for maintaining a temperature of the source material within a target range is selected using a look up table. For example, the look up table ties different duration times with different amounts of substance to be released. For example, the look up table ties different duration times with the types of substances to be delivered. For example, the look up table ties different duration times with one or more personal properties of the user and their optionally their expected inhalation duration, for example based on: age, sex, physical condition, condition being treated, etc.

In some embodiments, the device is programmed with pre-defined heating profiles, for example for different dosing regimens. For example, a first heating profile is set to maintain the source material within a target temperature range for a duration in the range 1100-1900 milliseconds, 1200-1600 milliseconds, 1000-1500 milliseconds or intermediate, longer or shorter duration.

In some embodiments, for maintaining the source material at the target temperature range, the heating element(s) may be heated and/or allowed to cool, optionally in cycles.

In some embodiments, the heating is reduced or terminated to then allow for cooling. In some embodiments, the airflow rate is increased to potentially accelerate cooling. In some embodiments, airflow from a different direction is added to potentially accelerate cooling (for example, airflow across the length of the source material unit).

Some examples of target temperature ranges, which in some embodiments are set to include a vaporization temperature of one or more selected substances, may include: a vaporization range of 150° C.+/−20° C. for THC from cannabis; a vaporization range of 160+/−20° C. for CBD from cannabis; a vaporization range of 250-350° C. for nicotine from tobacco.

Other examples of substances released from source material and their respective release conditions are for example as described in PCT publication WO2019/159170, titled “METHOD AND INHALER FOR PROVIDING TWO OR MORE SUBSTANCES BY INHALATION”, see for examples tables 1-5.

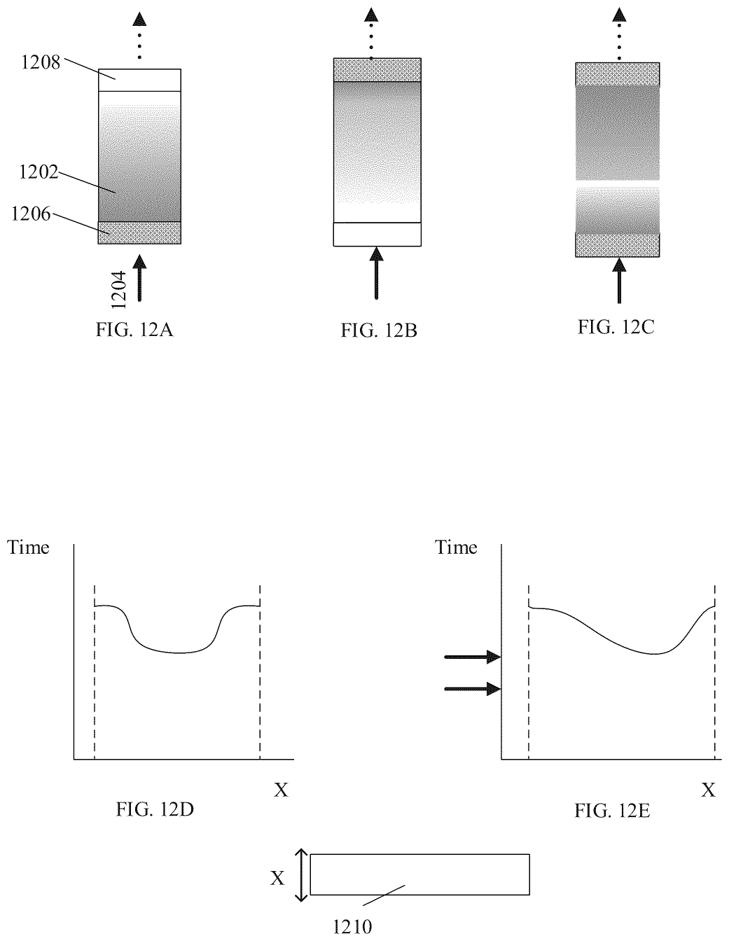

FIGS. 12A-C schematically illustrate an estimated effect of heating a source material pallet from one or two surfaces of the pallet, according to some embodiments. In some embodiments, heating is performed according to an expected heat distribution pattern within the source material. The expected pattern is, for some embodiments, deduced based on results of experimentation (such as temperature measurements performed in the lab).

In some embodiments, the heat distribution pattern in the source material pallet 1202 is affected by air flowing through the pallet, in this example in the direction indicated by arrow 1204.

FIG. 12A shows the effect of heating a heating element 1206 located upstream, according to some embodiments. The source material at layers adjacent the heated heating element are shown to reach a higher temperature than layers located downstream and closer to the opposing heating element 1208 (which, in this example, is not heated).

FIG. 12B shows the effect of heating the downstream heating element 1208. The source material layers adjacent element 1208 are shown to reach a higher temperature than layers located upstream, closer to heating element 1206. Due to the passing of air through the source material and the direction of flow, layers closer to element 1206 in this heating configuration are heated to a lower temperature as compared to layers closer to element 1208 in FIG. 12A.

FIG. 12C shows the effect of heating both heating elements 1206 and 1208 to a similar temperature. Layers adjacent both heating elements are optionally heated to a similar extent, but, due to the passing of air through the source material and the direction of flow, more central layers which are closer to the upstream end may have a reduced temperature relative to the surrounding layers. Therefore, due to the passing of airflow, even when heating both heating elements to a similar temperature there exists a temperature distribution pattern which is non-uniform and naturally not homogenous.

In view of the above, in some embodiments, heating of one or both of the heating elements is controlled taking into account an expected, optionally non-homogenous temperature distribution inside the source material.

In some embodiments, the temperature distribution pattern is used for prediction of an actual temperature distribution within the source material.

FIGS. 12D-E graphically compare heating of a source material pallet 1210 when there is air flowing through the pallet and when there is no airflow through the pallet, according to some embodiments. The graphs show a temperature distribution along dimension X of the pallet, representing the thickness of the pallet, over time.

In FIG. 12D, when no airflow is present, heating the pallet at the two opposite surfaces of the pallet generates, over time, a temperature distribution in which source material layers closer to the heating elements (indicated by the dashed lines) are potentially heated to a similar extent (assuming identical heating of the heating elements), while more central layers are heated to a lower extent.

In FIG. 12E, the presence of airflow entering through one side of the pallet and leaving through the opposite side changes the temperature distribution, for example due to cooling caused to layers adjacent the pallet side through which the air flows in.

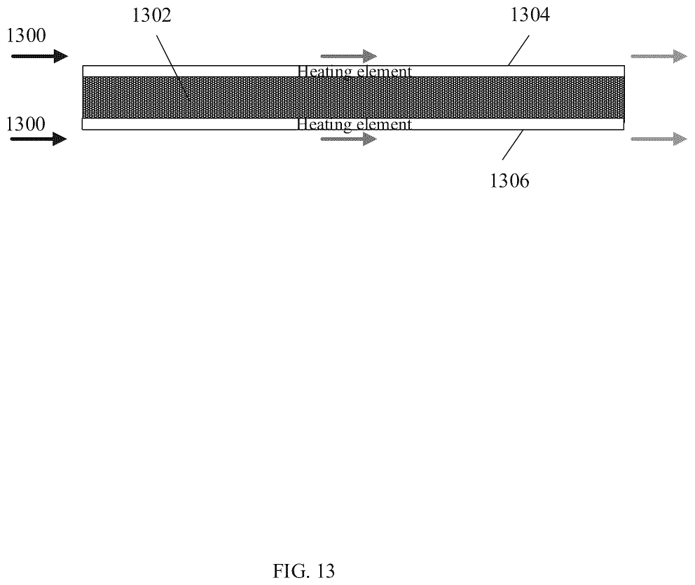

FIG. 13 is a schematic drawing of an airflow scheme across one or more surfaces of a source material pallet, according to some embodiments.

In some embodiments, flow of air is allowed and/or directed to pass along a long dimension of the pallet. In some embodiments, flow along a long dimension is in addition to flow along the short dimension (e.g. across the thickness of the pallet). Optionally, heating and/or airflow are controlled independently for each of the surfaces of the pallet. Optionally, heating of each of the heating elements is controlled separately. Optionally, airflow across each of the heating elements is controlled separately.

In some embodiments, flow is only along a long dimension of the pallet.

In some embodiments, airflow 1300 is directed to pass across surface(s) of the pallet 1302, for example, across a top surface and/or across a bottom surface of the pallet. In some embodiments, the flow of air is directed and/or allowed across a heating element 1304 and/or 1306. Optionally, the flow of air picks up vapors of the one or more substances released from the pallet, for example vapors released through apertures of a heating element (e.g. a heating element in the form of a mesh).

In some embodiments, a direction of airflow (e.g. along a horizontal axis of the pallet, from right to left or the opposite) is controlled. In some embodiments, the direction of flow along the horizontal axis is similar for both sides of the pallet. Alternatively, the direction of flow along the horizontal axis is different for each side of the pallet.

It is expected that during the life of a patent maturing from this application many relevant inhalers and/or source material units/dose cartridges will be developed and the scope of these terms is intended to include all such new technologies a priori.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, a “plurality” means two or more. As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to 10 be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A heating module useable in an inhaler device configured to receive a source material unit, the source material unit including first and second electrically resistive heating elements in contact with source material, the heating module comprising:
at least two electrical contacts shaped and positioned to engage the first and second electrically resistive heating elements of the source material unit when the source material unit is received within the inhaler device; and
circuitry for controlling conduction of current by the at least two electrical contacts for heating the first and second heating elements to raise a temperature of at least 85% of the source material to a target temperature; the circuitry configured to control heating of the first heating element to a first temperature and heating of the second heating element to a second temperature different than the first temperature.

2. A kit comprising:
an inhaler device including a heating module according to claim 1; and
a source material unit including first and second electrically resistive heating elements in contact with source material, the source material unit shaped and sized to be received within a housing of the inhaler.

3. (canceled)

4. The heating module according to claim 1, implemented in an inhaler device for delivery of one or more substances released from a source material to an inhaling user.

5. The heating module according to claim 1, wherein the circuitry is configured to control heating of the first and second heating elements to maintain the heated source material within a range of +/−15% of the target temperature.

6. The heating module according to claim 1, wherein the circuitry is configured to control heating of the first and second heating elements in accordance with a rate of airflow through the source material unit.

7. The heating module according to claim 6, wherein the airflow is directed and/or allowed across the heating element.

8. The heating module according to claim 6, wherein the flow of air carry vapors of the one or more substances released from the source material.

9. The heating module according to claim 1, comprising at least one sensor positioned to measure, when the source material unit is received within the inhaler device, the temperature of at least one of: the first heating element, the second heating element, the source material or portions; the circuitry configured to control heating of the first and second heating elements in response to an indication received from the at least one sensor.

10. The heating module according to claim 1, wherein the circuitry controls heating of the first and second heating elements to raise a temperature of the source material to a temperature range within 10° C. of a vaporization temperature of the at least one substance within less than 2 seconds.

11. The heating module according to claim 10, wherein the circuitry controls heating of the first and second heating elements to stabilize and maintain the source material temperature within in the vaporization temperature range for a time period of 0.5 seconds or longer.

12. The heating module according to claim 11, wherein the first and second heating elements are parts of a single heating element and wherein the circuitry is configured to deliver a similar amount of electric energy to both the first and second heating elements.

13. The heating module according to claim 1, wherein heating of the heating element to raise the temperature of at least 85% of the source material to said target temperature involves supplying power suitable to raise a temperature of said electrically resistive heating elements to said temperature.

14. The heating module according to claim 13, wherein the supplying power comprises taking into account parameters of the airflow, which may reduce the actual temperature of the heating element.

15. The kit according to claim 2, wherein the source material is in the form of a pallet.

16. The kit according to claim 15, wherein a thickness of the pallet is between 0.5-1 mm.

17. The kit according to claim 15, wherein a surface area of each of first and second opposing surfaces of the pallet which are heated by the first and second heating elements respectively is between 200-300 mm\hat{ }2.

18. The kit according to claim 15, wherein a weight of the pallet is between 100-150 mg.

19. The kit according to claim 15, wherein the pallet comprises source material particles dispersed with spaces therebetween through which air is allowed to flow.
